SEQUENCE LISTING (53 sequences)

SEQ ID NO: 1 - protein, 346 aa, Homo sapiens [sequence omitted]

SEQ ID NO: 2 - protein, 96 aa, Homo sapiens
MDSSGPKHLNMKLTRAQFEGIVTDLIRRTIAPCQKAMQDAEVSKSDIGEVILVGGMTRMPKVQQTVQDLFGRAPSKAVNPDEAVAIGAAIQGGVLA

SEQ ID NO: 3 - protein, 150 aa, Homo sapiens
MDSSGPKHLNMKLTRAQFEGIVTDLIRRTIAPCQKAMQDAEVSKSDIGEVILVGGMTRMPKVQQTVQDLFGRAPSKAVNPDEAVAIGAAIQGGVLAGDVTDVLLLDVTPLSLGIETLGGVFTKLINRNTTIPTKKSQVFSTAADGQTQVE

SEQ ID NO: 4 - protein, 245 aa, Homo sapiens
MDSSGPKHLNMKLTRAQFEGIVTDLIRRTIAPCQKAMQEVSKSDIGEVILVGGMTRMPKVQQTVQGLFGRAPSKAVNPDEAVAIGAAIQGGVLAGDVTDVLLLDVTPLSLGIETLGGVFTKLINRNTTIPTKKSQVFSTAADGQTQVEIKVCQGEREMAGDNKLLGQFTLIGIPPAPRGVPQIEVTFDIDANGIVHVSAKDKGTGRERQIVIQSSGGLSKDDIEENMVKNAEKYAEEDRRKKERV

SEQ ID NO: 5 - protein, 60 aa, Homo sapiens
LAGDVTDVLLLDVTPLSLGIETLGGVFTKLINRNTTIPTKKSQVFSTAADGQTQVEIKVC

SEQ ID NO: 6 - protein, 154 aa, Homo sapiens
VLAGDVTDVLLLDVTPLSLGIETLGGVFTKLINRNTTIPTKKSQVFSTAADGQTQVEIKVCQGEREMAGDNKLLGQFTLIGIPPAPRGVPQIEVTFDIDANGIVVHVSAKDKGTGRERQIVIQSSGGLSKDDIENMVKNAEKYAEEDRRKKERV

SEQ ID NO: 7 - protein, 48 aa, Homo sapiens
QDAEVSKSDIGEVILVGGMTRMPKVQQTVQDLFGRAPSKAVNPDEAVA

SEQ ID NO: 8 - DNA, 180 bp, Homo sapiens
ttggccggcgatgtcacggatgtgctgctccttgatgtcactcccctgtctctgggtattgaaactctaggaggtgtctttaccaaacttattaataggaataccactattccaaccaagaagagccaggtattctctactgccgctgatggtcaaacgcaagtggaaattaaagtgtgt

SEQ ID NO: 9 - protein, 49 aa, Homo sapiens
LAGDVTDVLLLDVTPLSLGIETLGGVFTKLINRNTTIPTKKSQVFSTAA

SEQ ID NO: 10 - protein, 36 aa, Homo sapiens
LAGDVTDVLLLDVTPLSLGIETLGGVFTKLINRNTT

SEQ ID NO: 11 - protein, 24 aa, Homo sapiens
LAGDVTDVLLLDVTPLSLGIETLG

SEQ ID NO: 12 - protein, 15 aa, Homo sapiens
LAGDVTDVLLLDVTP

SEQ ID NO: 13 - protein, 45 aa, Homo sapiens
PLSLGIETLGGVFTKLINRNTTIPTKKSQVFSTAADGQTQVEIKV

SEQ ID NO: 14 - protein, 35 aa, Homo sapiens
PLSLGIETLGGVFTKLINRNTTIPTKKSQVFSTAA

SEQ ID NO: 15 - protein, 22 aa, Homo sapiens
PLSLGIETLGGVFTKLINRNTT

SEQ ID NO: 16 - protein, 10 aa, Homo sapiens
PLSLGIETLG

SEQ ID NO: 17 - protein, 37 aa, Homo sapiens
GGVFTKLINRNTTIPTKKSQVFSTAADGQTQVEIKVC

SEQ ID NO: 18 - protein, 26 aa, Homo sapiens
GGVFTKLINRNTTIPTKKSQVFSTAA

SEQ ID NO: 19 - protein, 12 aa, Homo sapiens
GGVFTKLINRNT

SEQ ID NO: 20 - protein, 26 aa, Homo sapiens
NTTIPTKKSQVFSTAADGQTQVEIKV

SEQ ID NO: 21 - protein, 15 aa, Homo sapiens
NTTIPTKKSQVFSTA

SEQ ID NO: 22 - protein, 10 aa, Homo sapiens
GQTQVEIKVC

SEQ ID NO: 23 - DNA, 33 bp, Homo sapiens
tagggatccttggccggcgatgtcacggatgtg

SEQ ID NO: 24 - DNA, 29 bp, Homo sapiens
tagggatcccccctgtctctgggtattga

SEQ ID NO: 25 - DNA, 38 bp, Homo sapiens
tagggatccggaggtgtctttaccaaacttattaatag

SEQ ID NO: 26 - DNA, 35 bp, Homo sapiens
tagggatccaataccactattccaaccaagaagag

SEQ ID NO: 27 - DNA, 29 bp, Homo sapiens
tagggatccggtcaaacgcaagtggaaat

SEQ ID NO: 28 - DNA, 32 bp, Homo sapiens
ctagaattctcagggagtgacatcaaggagca

SEQ ID NO: 29 - DNA, 37 bp, Homo sapiens
ctagaattctcatcctagagtttcaatacccagagac

SEQ ID NO: 30 - DNA, 42 bp, Homo sapiens
ctagaattctcaagtggtattcctattaataagtttggtaaa

SEQ ID NO: 31 - DNA, 33 bp, Homo sapiens
ctagaattctcaagcggcagtagagaatacctg

SEQ ID NO: 32 - DNA, 146 bp, Homo sapiens
ttggccggcgatgtcacggatgtgctgctccttgatgtcactcccctgtctctgggtattgaaactctaggaggtgtctttaccaaacttattaataggaataccactattccaaccaagaagagccaggtattctctactgccgc

SEQ ID NO: 33 - DNA, 108 bp, Homo sapiens
ttggccggcgatgtcacggatgtgctgctccttgatgtcactcccctgtctctgggtattgaaactctaggaggtgtctttaccaaacttattaataggaataccact

SEQ ID NO: 34 - DNA, 72 bp, Homo sapiens
ttggccggcgatgtcacggatgtgctgctccttgatgtcactcccctgtctctgggtattgaaactctagga

SEQ ID NO: 35 - DNA, 45 bp, Homo sapiens
ttggccggcgatgtcacggatgtgctgctccttgatgtcactccc

SEQ ID NO: 36 - DNA, 138 bp, Homo sapiens
cccctgtctctgggtattgaaactctaggaggtgtctttaccaaacttattaataggaataccactattccaaccaagaagagccaggtattctctactgccgctgatggtcaaacgcaagtggaaattaaagtgtgt

SEQ ID NO: 37 - DNA, 105 bp, Homo sapiens
cccctgtctctgggtattgaaactctaggaggtgtctttaccaaacttattaataggaataccactattccaaccaagaagagccaggtattctctactgccgct

SEQ ID NO: 38 - DNA, 66 bp, Homo sapiens
cccctgtctctgggtattgaaactctaggaggtgtctttaccaaacttattaataggaataccact

SEQ ID NO: 39 - DNA, 30 bp, Homo sapiens
cccctgtctctgggtattgaaactctagga

SEQ ID NO: 40 - DNA, 111 bp, Homo sapiens
ggaggtgtctttaccaaacttattaataggaataccactattccaaccaagaagagccaggtattctctactgccgctgatggtcaaacgcaagtggaaattaaagtgtgt

SEQ ID NO: 41 - DNA, 78 bp, Homo sapiens
ggaggtgtctttaccaaacttattaataggaataccactattccaaccaagaagagccaggtattctctactgccgct

SEQ ID NO: 42 - DNA, 39 bp, Homo sapiens
ggaggtgtctttaccaaacttattaataggaataccact

SEQ ID NO: 43 - DNA, 81 bp, Homo sapiens
aataccactattccaaccaagaagagccaggtattctctactgccgctgatggtcaaacgcaagtggaaattaaagtgtgt

SEQ ID NO: 44 - DNA, 48 bp, Homo sapiens
aataccactattccaaccaagaagagccaggtattctctactgccgct

SEQ ID NO: 45 - DNA, 30 bp, Homo sapiens
ggtcaaacgcaagtggaaattaaagtgtgt

SEQ ID NO: 46 - protein, 60 aa, Homo sapiens
GDVKDVLLLDVTPLSLGIETMGGVMTTLIAKNTTIPTKHSQVFSTAEDNQSAVTIHVLQG

SEQ ID NO: 47 - DNA, 1041 bp, Homo sapiens
atggattcttctggacccaagcatttgaatatgaagttgacccgtgctcaatttgaagggattgtcactgatctaatcagaaggactatcgctccatgccaaaaagctatgcaagatgcagaagtcagcaagagtgacataggagaagtgattcttgtgggtggcatgactaggatgcccaaggttcagcagactgtacaggatctttttggcagagccccaagtaaagctgtcaatcctgatgaggctgtggccattggagctgccattcagggaggtgtgttggccggcgatgtcacggatgtgctgctccttgatgtcactcccctgtctctgggtattgaaactctaggaggtgtctttaccaaacttattaataggaataccactattccaaccaagaagagccaggtattctctactgccgctgatggtcaaacgcaagtggaaattaaagtgtgtcagggtgaaagagagatggctggagacaacaaactccttggacagtttactttgattggaattccaccagcccctcgtggagttcctcagattgaagttacatttgacattgatgccaatgggatagtacatgtttctgctaaagataaaggcacaggacgtgagcagcagattgtaatccagtcttctggtggattaagcaaagatgatattgaaaatatggttaaaaatgcagagaaatatgctgaagaagaccggcgaaagaaggaacgagttgaagcagttaatatggctgaaggaatcattcacgacacagaaaccaagatggaagaattcaaggaccaattacctgctgatgagtgcaacaagctgaaagaagagatttccaaaatgagggagctcctggctagaaaagacagcgaaacaggagaaaatattagacaggcagcatcctctcttcagcaggcatcactgaagctgttcgaaatggcatacaaaaagatggcatctgagcgagaaggctctggaagttctggcactggggaacaaaaggaagatcaaaaggaggaaaaacagtaa

SEQ ID NO: 48 - DNA, 1917 bp, Escherichia coli
atgggtaaaataattggtatcgacctgggtactaccaactcttgtgtagcgattatggatggcaccactcctcgtgtgctggagaacgccgaaggcgatcgcaccacgccttctatcattgcctatacccaggatggtgaaactctggttggtcagccggctaaacgtcaggcagtgacgaacccgcaaaacaccctgtttgcgattaaacgcctgattggccgccgcttccaggacgaagaagtacagcgtgatgtttccatcatgccgttcaaaattattgctgctgataacggcgacgcatgggtcgaagttaaaggccagaaaatggcaccgccgcagatctctgctgaagtgctgaaaaaaatgaagaaaaccgctgaagattacctgggtgaaccggtaactgaagctgttatcaccgtaccggcatactttaacgatgctcagcgtcaggcaaccaaagacgcaggccgtatcgctggtctggaagtaaaacgtatcatcaacgaaccgaccgcagctgcgctggcttacggtctggacaaaggtactggcaaccgtactatcgcggtttatgacctgggtggtggtactttcgatatttctattatcgaaatcgacgaagttgacggcgaaaaaaccttcgaagttctggcaaccaacggtgatacccacctgggtggtgaagacttcgacagccgtctgatcaactacctggttgaagaattcaagaaagatcagggcattgacctgcgcaacgatccgctggcaatgcagcgcctgaaagaagcggcagaaaaagcgaaaatcgaactgtcttccgctcagcagaccgacgttaacctgccgtacatcactgcagatgcgaccggtccgaaacacatgaacatcaaagtgactcgtgcgaaactggaaagcctggttgaagatctggtaaaccgttccattgagccgctgaaagttgcactgcaggacgctggcctgtccgtatctgatatcgacgacgttatcctcgttggtggtcagactcgtatgccaatggttcagaagaaagttgctgagttctttggtaaagagccgcgtaaagacgttaacccggacgaagctgtagcaatcggtgctgctgttcagggtggtgttctgactggtgacgtgaaagacgtactgctgctggacgttaccccgctgtctctgggtatcgaaaccatgggcggtgtgatgacgacgctgatcgcgaaaaacaccactatcccgaccaagcacagccaggtgttctctaccgctgaagacaaccagtctgcggtaaccatccatgtgctgcagggtgaacgtaaacgtgcggctgataacaaatctctgggtcagttcaacctggatggtatcaacccggcaccgcgcggcatgccgcagatcgaagttaccttcgatatcgatgctgacggtatcctgcacgtttccgcgaaagataaaaacagcggtaaagagcagaagatcaccatcaaggcttcttctggtctgaacgaagatgaaatccagaaaatggtacgcgacgcagaagctaacgccgaagctgaccgtaagtttgaagagctggtacagactcgcaaccagggcgaccatctgctgcacagcacccgtaagcaggttgaagaagcaggcgacaaactgccggctgacgacaaaactgctatcgagtctgcactgactgcactggaaactgctctgaaaggtgaagacaaagccgctatcgaagcgaaaatgcaggagctggcacaggtttcccagaaactgatggaaatcgcccagcagcaacatgcccagcagcagactgccggtgctgatgcttctgcaaacaacgcgaaagatgacgatgttgtcgacgctgaatttgaagaagtcaaagacaaaaaataa

SEQ ID NO: 49 - protein, 638 aa, Escherichia coli
MGKIIGIDLGTTNSCVAIMDGTTPRVLENAEGDRTTPSIIAYTQDGETLVGQPAKRQAVTNPQNTLFAIKRLIGRRFQDEEVQRDVSIMPFKIIAADNGDAWVEVKGQKMAPPQISAEVLKKMKKTAEDYLGEPVTEAVITVPAYFNDAQRQATKDAGRIAGLEVKRIINEPTAAALAYGLDKGTGNRTIAVYDLGGGTFDISIIEIDEVDGEKTFEVLATNGDTHLGGEDFDSRLINYLVEEFKKDQGIDLRNDPLAMQRLKEAAEKAKIELSSAQQTDVNLPYITADATGPKHMNIKVTRAKLESLVEDLVNRSIEPLKVALQDAGLSVSDIDDVILVGGQTRMPMVQKKVAEFFGKEPRKDVNPDEAVAIGAAVQGGVLTGDVKDVLLLDVTPLSLGIETMGGVMTTLIAKNTTIPTKHSQVFSTAEDNQSAVTIHVLQGERKRAADNKSLGQFNLDGINPAPRGMPQIEVTFDIDADGILHVSAKDKNSGKEQKITIKASSGLNEDEIQKMVRDAEANAEADRKFEELVQTRNQGDHLLHSTRKQVEEAGDKLPADDKTAIESALTALETALKGEDKAAIEAKMQELAQVSQKLMEIAQQQHAQQQTAGADASANNAKDDDVVDAEFEEVKDKK

SEQ ID NO: 50 - DNA, 288 bp, Homo sapiens
atggattcttctggacccaagcatttgaatatgaagttgacccgtgctcaatttgaagggattgtcactgatctaatcagaaggactatcgctccatgccaaaaagctatgcaagatgcagaagtcagcaagagtgacataggagaagtgattcttgtgggtggcatgactaggatgcccaaggttcagcagactgtacaggatctttttggcagagccccaagtaaagctgtcaatcctgatgaggctgtggccattggagctgccattcagggaggtgtgttggcc

SEQ ID NO: 51 - DNA, 450 bp, Homo sapiens
atggattcttctggacccaagcatttgaatatgaagttgacccgtgctcaatttgaagggattgtcactgatctaatcagaaggactatcgctccatgccaaaaagctatgcaagatgcagaagtcagcaagagtgacataggagaagtgattcttgtgggtggcatgactaggatgcccaaggttcagcagactgtacaggatctttttggcagagccccaagtaaagctgtcaatcctgatgaggctgtggccattggagctgccattcagggaggtgtgttggccggcgatgtcacggatgtgctgctccttgatgtcactcccctgtctctgggtattgaaactctaggaggtgtctttaccaaacttattaataggaataccactattccaaccaagaagagccaggtattctctactgccgctgatggtcaaacgcaagtggaa

SEQ ID NO: 52 - DNA, 738 bp, Homo sapiens
atggattcttctggacccaagcatttgaatatgaagttgacccgtgctcaatttgaagggattgtcactgatctaatcagaaggactatcgctccatgccaaaaagctatgcaagatgcagaagtcagcaagagtgacataggagaagtgattcttgtgggtggcatgactaggatgcccaaggttcagcagactgtacaggatctttttggcagagccccaagtaaagctgtcaatcctgatgaggctgtggccattggagctgccattcagggaggtgtgttggccggcgatgtcacggatgtgctgctccttgatgtcactcccctgtctctgggtattgaaactctaggaggtgtctttaccaaacttattaataggaataccactattccaaccaagaagagccaggtattctctactgccgctgatggtcaaacgcaagtggaaattaaagtgtgtcagggtgaaagagagatggctggagacaacaaactccttggacagtttactttgattggaattccaccagcccctcgtggagttcctcagattgaagttacatttgacattgatgccaatgggatagtacatgtttctgctaaagataaaggcacaggacgtgagcagcagattgtaatccagtcttctggtggattaagcaaagatgatattgaaaatatggttaaaaatgcagagaaatatgctgaagaagaccggcgaaagaaggaacgagtt

SEQ ID NO: 53 - DNA, 459 bp, Homo sapiens
gtgttggccggcgatgtcacggatgtgctgctccttgatgtcactcccctgtctctgggtattgaaactctaggaggtgtctttaccaaacttattaataggaataccactattccaaccaagaagagccaggtattctctactgccgctgatggtcaaacgcaagtggaaattaaagtgtgtcagggtgaaagagagatggctggagacaacaaactccttggacagtttactttgattggaattccaccagcccctcgtggagttcctcagattgaagttacatttgacattgatgccaatgggatagtacatgtttctgctaaagataaaggcacaggacgtgagcagcagattgtaatccagtcttctggtggattaagcaaagatgatattgaaaatatggttaaaaatgcagagaaatatgctgaagaagaccggcgaaagaaggaacgagtt

(19) United States
(12) Patent Application Publication
(10) Pub. No.: US 2016/0052980 A1
(43) Pub. Date: Feb. 25, 2016

(54) CRP40 FRAGMENTS FOR THE TREATMENT OF NEUROLOGICAL DISORDERS

(71) Applicant: CRP40 INC., Ottawa (CA)

(72) Inventors: Joseph GABRIELE, Stoney Creek (CA); Ram MISHRA, Dundas (CA); Zdenek PRISTUPA, Caledon (CA)

(21) Appl. No.: 14/782,141

(22) PCT Filed: Apr. 4, 2014
(86) PCT No.: PCT/CA2014/050349

§ 371 Date: Oct. 2, 2015

Related U.S. Application Data
(60) Provisional application No. 61/808,398, filed on Apr. 4, 2013.
(60) Provisional application No. 61/932,460, filed on Jan. 28, 2014.

(51) Int. Cl. C07K 14/47; C07K 5/10; C07K 7/08; C07K 5/08; C07K 16/18; C07K 7/06

(52) U.S. Cl. CPC C07K 14/4702; C07K 16/18; C07K 7/06; C07K 7/08; C07K 5/08; C07K 5/10; A61K 38/00

(57) ABSTRACT

Disclosed herein are human CRP40 fragments and polynucleotides encoding them. The CRP40 fragments and polynucleotides may be useful in the treatment of diseases associated with one or more of oxidative stress, mitochondrial dysfunction and abnormal protein folding, including various neurological disorders.

### CROSS REFERENCE TO RELATED APPLICATIONS

This application claims the benefit of priority of U.S. Provisional Patent Application No. 61/808,398, filed Apr. 4, 2013, and U.S. Provisional Patent Application No. 61/932,460, filed Jan. 28, 2014, which are incorporated herein by reference in their entirety.

### FIELD OF THE DISCLOSURE

Human catecholamine-regulated protein, CRP40, is a 40 kDa molecular chaperone protein identified by the present inventors. The present disclosure relates to novel fragments of human CRP40 and polynucleotides encoding them. More particularly, the present disclosure relates to novel fragments of CRP40 comprising at least a portion of a putative substrate binding region that is not involved in dopamine binding.

### BACKGROUND

Catecholamines are neurotransmitters and/or hormones found in the peripheral systems of the body as well as the central nervous system (CNS). They consist of dopamine (DA), norepinephrine and epinephrine, which are synthesized from the amino acid tyrosine. DA exerts its physiological actions by acting on a family of related G protein-coupled receptors. DA receptors are divided into two major groups and include D1 and D2 dopamine receptor classes. This classification is based on the finding that the D1 receptor class, but not the D2 receptor class, has the ability to couple to adenylyl cyclase (AC) and modulate cyclic adenosine monophosphate (cAMP) production. The functions of the different classes of DA receptors have been extensively studied. The various DA receptor subtypes in the brain are known to participate in locomotion, attention, decision making, impulse control, motor learning, reproductive behaviour and regulation of food intake (Rondou et al., 2010).

Numerous studies have focused on the impact of DA cytotoxicity in neurodegenerative diseases, as well as other impairments including mitochondrial dysfunction, oxidative stress and aberrations to the ubiquitin-proteosome systems (Miyazaki and Asanuma, 2009). DA is usually stable within synaptic vesicles, however, it may become cytotoxic following damage to DA neurons. This process may lead to the oxidation of excess cytosolic DA or _{L}-DOPA to superoxide anions, DA quinones, or DOPA quinones. The damage to proteins by DA quinones and other reactive metabolites may be potentiated by dysfunctional protective mechanisms, which may involve molecular chaperone proteins.

Dysregulation of the dopaminergic systems in the brain has been linked to multiple neurological and neuropsychiatric disorders, including Parkinson's disease (PD), schizophrenia, depression, attention-deficit hyperactivity disorder, Huntington's disease, and Tourette's syndrome (Beaulieu and Gainetdinov, 2011). One of the earliest disorders related to DA-dysfunction was PD, which is a progressive neurodegenerative disorder characterized by various motor and non-motor clinical symptoms. The cardinal pathological features of PD include the selective degeneration of the nigrostriatal pathway, loss of dopaminergic neurons, and the appearance of intraneuronal inclusions, known as Lewy bodies, in surviving neurons.

The etiology of PD is still incompletely understood. This has led to significant challenges in diagnosis of PD and in development of sustainable therapeutics for PD. _{L}-DOPA has been the mainstay of PD therapy for the past four decades. While initially being able to compensate for the loss of dopaminergic neurons, _{L}-DOPA fails to halt disease progression and eventually leads to motor complications as well as psychiatric problems such as hallucinations and delirium (Obeso et al., 2010). The development of DA agonists, which are used in early stages of the disease, along with atypical neuroleptics (e.g., clozapine), has somewhat alleviated these problems and improved the therapeutic outcomes of PD patients. However, many these drugs have negative side effects. There is a need for new and improved therapies for PD and other neurological disorders.

Researchers have not been able to determine a single cause for PD thus far. Most cases seem to be idiopathic. PD has been linked to oxidative stress, genetic mutations, mitochondrial dysfunction, and protein misfolding (Schapira, 2009; Schapira and Jenner, 2011). It is estimated that 2-3% of late-onset cases and about 50% of the early-onset cases of PD are linked to genetic mutations (Farrer, 2006). Environmental factors may contribute to the vulnerability of the substantia nigra to cell death and degeneration by causing mitochondrial dysfunction and oxidative stress. As well, abnormalities in protein function seen in PD may be caused by defective molecular chaperone proteins.

Molecular chaperone proteins are essential for multiple cellular functions including protein quality control and maintenance of protein homeostasis. In the cell, proteins are susceptible to changes in conformation which may alter their function and cause them to aggregate into cytotoxic complexes. Molecular chaperones may function by preventing protein aggregation using adenosine triphosphate (ATP) as energy to drive conformational changes that are necessary for refolding their particular targets (Soti et al., 2005); solubilizing initial protein aggregates and aiding in the folding of nascent proteins as they are synthesized by ribosomes (Ellis, 1987); and controlling protein-protein interactions by influencing conformational changes. In cases where cellular proteins are extensively damaged and where molecular chaperones cannot help to refold the proteins, they may function in targeting proteins for degradation by facilitating their transfer to proteolytic systems or sequestering the damaged proteins into larger aggregates (Soti et al., 2005).

Numerous classes of molecular chaperones have been described thus far. Many of these proteins are known as heat-shock proteins (Hsp) or stress proteins because it has been found that various cellular stresses upregulate their expression. Different families of molecular chaperone proteins are classified according to their molecular weights and include the Hsp40, Hsp60, Hsp70, Hsp90, Hsp100, and small Hsp proteins (Kiang and Tsokos, 1998).

The heat-shock protein 70 kDa (Hsp70) family represents one of the most ubiquitous classes of molecular chaperones. These proteins are highly conserved and may be constitutively expressed or upregulated by various stressors (Chang et al., 2007). The Hsp70 family plays a role in assisting in de novo folding of proteins as well as protein trafficking and targeting of misfolded proteins for proteolytic degradation (Chang et al., 2007). They function in collaboration with the Hsp40 or DnaJ family of proteins as well as various nucleotide-exchange factors in ATP-driven reactions (Mayer et al., 2000). Hsp70 assist in de novo folding of proteins by binding to nascent chains through allosteric coupling of its N-terminal ATPase domain (˜40 kDa) with the peptide-binding domain (˜25 kDa) which is located at the C-terminal region (Zhu et al., 1996). The C-terminal domain consists of a β-sandwich subdomain that recognizes hydrophobic segments of approximately 7 amino acid residues and an α-helical lid which regulates ATP-binding (Hartl and Hayer-Hartl, 2009).

Molecular chaperone proteins are emerging as important factors in a number of human disorders, such as PD, Alzheimer's, Huntington's and cerebral ischemia which share the common characteristics of aberrant protein folding. Molecular chaperone proteins are an interesting target for research in the realm of neurodegenerative disorders due to their diverse functionalities and roles in cells.

Mortalin (also known as mortalin-2 or mitochondrial heat-shock protein 70) is an essential ubiquitously expressed molecular chaperone with multiple roles which include participating in mitochondrial biogenesis, maintaining mitochondrial protein integrity, and aiding in import of mitochondrial proteins into the matrix via formation of ATP-dependent motors (Deocaris et al., 2008). Mortalin consists of 649 amino acids and is encoded by the nuclear gene HSPA9B located on chromosome 5q31.1.1 (Kaul et al., 2007).

Evidence for the involvement of mortalin in human diseases has been accumulating over the past decade. Mortalin has been linked to diseases such as PD, Schizophrenia, Alzheimer's and Huntington's (Deocaris et al., 2008; Gabriele et al., 2010). Mortalin has been implicated in neurogenesis and neurodegeneration processes (Deocaris et al., 2008). It is suggested that mortalin may serve a protective function in ischemia by limiting the accumulation of reactive oxygen species (ROS) in neurons (Liu et al., 2005). Since mortalin has been found to exert various cytoprotective functions that permit cell survival under stressful conditions, it has been implicated in cancer and aging systems (Kaul et al., 2007). Mortalin has been implicated in several neurodegenerative diseases such as Alzheimer's disease, and PD, which are thought to be associated with old age. As well, these diseases are associated with abnormal polypeptides which can form insoluble neurotoxic protein aggregates leading to cell death (Kaul et al., 2007). Also, there are notable defects in ubiquitin-proteosome degradation systems and responses to oxidative stress (Kaul et al., 2007). Osorio and colleagues (2007) showed differential expression of mortalin isoforms in hippocampi of Alzheimer's patients. In another study using an animal model of Alzheimer's disease, the ApoE knockout mouse model, it was shown that mortalin sustained Alzheimer's-associated oxidative damage, suggesting the involvement of this protein in the pathogenesis of this disease (Choi et al., 2004).

Jin and colleagues (2006) demonstrated that mortalin expression was significantly reduced in the SNc of PD patients. As well, in vitro experiments with MES cells, which express features of dopaminergic neurons, showed a significant decrease in mortalin levels when the cells were exposed to the mitochondrial Complex 1 inhibitor rotenone (Deocaris et al., 2008). Further in vitro studies by Van Laar and colleagues (2008) showed that treatment of PC12 cells with a DA quinone resulted in degeneration of dopaminergic terminals and a decrease in mortalin levels as well as reduction of mitochondrial proteins that may play important roles in PD. Recent studies by Shi and colleagues (2008) showed decreased levels of mortalin levels in mitochondria from post-mortem PD patients' substantia nigra brain samples. It is suggested that these reductions in mortalin may be a result of oxidative modification and protein damage that occurs in PD. Mortalin has even been found to associate with DJ-1, a gene responsible for a familial form of PD, and translocate to mitochondria in response to oxidative stress (Li et al., 2005). These observations were further confirmed in studies by Jin and colleagues (2007), who found that mortalin associates with not only DJ-1, but α-synuclein as well. These observations are significant because it is known that α-synuclein causes certain forms of sporadic PD.

Studies using 6-OHDA animal models of PD have also linked mortalin as an important factor in the disease progress. Weiss and colleagues (2006) injected human umbilical cord matrix stem cells into the striatum of 6-OHDA lesioned rats and observed that this manipulation was able to elicit complete alleviation of rotational symptoms that were previously induced by injection of apomorphine. Interestingly, upon proteonomic analysis, the authors found that mortalin was highly expressed in the umbilical cord matrix stem cells, lending support to the involvement of mortalin in PD pathology (Weiss et al., 2006).

Despite the crucial roles of mortalin that have been demonstrated in these studies, it is difficult to conjure up a possible therapeutic role of mortalin in neurological diseases due to its functions in cell cycle regulation and its evident association with human cancers. It has been suggested that mortalin may have the potential of causing cellular proliferation through its interaction with the tumour suppressor protein (p53) via its N-terminal domain (Deocaris et al., 2008). Mortalin expression was found to be elevated in many human cancers including brain cancer, colon cancer and leukemia (Kaul et al., 2007).

Early studies from the inventor's laboratory on catecholamine-regulated proteins reported the presence of three distinct brain-specific chaperone-like proteins with molecular weights of 26, 40, and 47 kDa, which were isolated based on their ability to bind DA and structurally related catecholamines (Ross et al., 1993; Ross et al., 1995). Further investigation confirmed the novel nature of these proteins as pharmacological and biochemical studies did not reveal similarities to other known catecholamine binding proteins or receptors in the brain (Modi et al., 1996). In 2001, Nair and Mishra cloned CRP40 from the bovine brain (GenBank #AF047009) and it was found that the novel protein shares significant structural homology with the human Hsp70 family of proteins including Hsp70 or mortalin (Genbank #BC024034) and Hsc70 (GenBank #NM006389) as well as proteins from other species including the rat and SH-SY5Y cells. Further, immunolocalization studies using SH-SY5Y cells revealed CRP40 colocalization with DA in vesicles (Nair and Mishra, 2001). Indeed, CRP40 has functional specificity for catecholamines as it associates with DA, epinephrine, and norepinephrine but not with other amines such as serotonin.

Nair and Mishra (2001) also revealed that bovine CRP40 expression is induced following exposure to heat-shock in SH-SY5Y cells, as seen with Hsp70. Further, CRP40 expression was also increased when SH-SY5Y cells were treated with excess DA, suggesting that CPR40 may share crucial properties with other heat-shock proteins such as mortalin, including protective roles in oxidative stress. These hypotheses were further strengthened by the fact that treatment of cells with CRP40 following heat-shock resulted in decreased protein denaturation and aggregation compared to non-treated controls. As well, immunofluorescence analyses showed that exposure of SH-SY5Y cells to detrimental DA oxidation caused CRP40 translocation to the nucleus, further implicating CRP40 as a protective, catecholamine-specific heat-shock protein.

In early studies to determine the specific localization of CRP40 in the brain, Ross and colleagues (1995) showed that the concentration of catecholamine-regulated proteins in the brain was the greatest at the striatum, followed by nucleus accumbens, olfactory tubercle, frontal cortex, hypothalamus, hippocampus, and lastly the cerebellum. Interestingly, catecholamine-regulated protein labelling studies on other non-CNS tissues such as skin, muscle, heart, liver, kidney, and spleen did not indicate the presence of these proteins in these areas. Goto and colleagues (2010) detected the CRP40 protein in the majority of SNc and striatal neurons. As well, CRP40 was found to colocalize with TH, the rate limiting enzyme in DA synthesis. These observations suggest that CRP40 may mediate a variety of functions in the nervous system and may be a key player in dopaminergically-driven diseases such as PD through its modulation of DA.

In 2009, Gabriele and colleagues cloned and characterized CRP40 from the human brain. It was found that the human 40 kDa CRP40 belongs to a family of heat-shock proteins and is a splice variant of mortalin. The human CRP40 displays 100% homology to human mortalin and is expressed from downstream exonic sequences of mortalin (e.g., exons 10-17). Bioinformatic analysis indicated that a promoter region for CRP40 may be contained at intron 9 of mortalin and that the human CRP40 is expressed from the HSPA9 gene (Gabriele et al., 2009). Further studies are required to identify the specific transcription factors and regulatory components necessary for CRP40 expression.

Gabriele and colleagues (2009) also discovered that the human CRP40 shares similar functions to the bovine CRP40 protein. Specifically, CRP40 was found to possess the following chaperone and catecholamine function characteristics: 1) CRP40 prevented thermal aggregation of firefly luciferase, suggesting its ability to protect cells from oxidative stress; 2) Overexpression of CRP40 in heat-shocked cells decreased protein denaturation and aggregation and increased cellular viability, indicating molecular chaperone-like functions; 3) CRP40 was found to bind the catecholamine DA with a low affinity and high capacity profile, which is characteristic of molecular chaperones involved in maintaining cellular protein homeostasis.

Several studies were performed in order to elucidate the function of the human CPR40. Gabriele and colleagues (2009) showed that treatment of cells with a D₂ receptor antagonist commonly used for Schizophrenia (e.g., haloperidol) caused the predicted increase in free synaptic DA, along with modulation of CRP40 levels, which increased significantly following haloperidol treatment in comparison to untreated cells. These results were also consistent with those observed in animal models. Sharan and colleagues (2001) showed differential modulation of CRP40 by DA D1 and D2 receptor antagonists following chronic treatment of rats with haloperidol and a D1 receptor antagonist, SCH23390. They found that haloperidol treatment induced marked increases in striatal CPR40, while treatment with the D1 receptor antagonist caused a decrease in striatal CPR40 (Sharan et al., 2001). These studies suggested that the CRP40 is differentially modulated by DA receptor antagonists.

Subsequent studies also discovered that CRP40 may be modulated by DA receptor agonists and psychotropic drugs. Gabriele and colleagues (2002) found that chronic, but not acute, d-amphetamine treatment of rats increased the expression of CRP40 in the striatum and the nucleus accumbens brain areas. Furthermore, when the expression levels of Hsp70 were examined following the same experiments, no significant changes in Hsp70 expression were observed, indicating that the modulatory effects of d-amphetamine were specific to the CPR40 protein (Gabriele et al., 2002). These findings were supported by studies examining the differential effects of cocaine treatment on CPR40 expression versus Hsp70 expression, which again showed specificity towards the CPR40 protein. Sharan and colleagues (2003) found that acute cocaine treatment increased CRP40 expression in the nucleus accumbens and striatum of rats, whereas chronic cocaine treatment increased CRP40 expression in the nucleus accumbens only. In contrast, cocaine treatment did not affect Hsp70 levels (Sharan et al., 2003). Further, it was hypothesized that the rise in CRP40 levels was due to increased protein synthesis, and not protein translocation, since pre-treatment with a protein synthesis inhibitor (e.g., anisomycin) inhibited the increase in CRP40 levels following cocaine treatment (Sharan et al., 2003). Increased CRP40 expression in response to cocaine, which induces oxidative stress, indicates that this protein may play a neuroprotective role in the brain.

The differential effects on CRP40 expression in the nucleus accumbens following acute and chronic treatment regimens were further examined by Gabriele and colleagues (2007). It was shown that chronic, but not acute, haloperidol treatment induced an increase in CPR40 levels in the nucleus accumbens core region (e.g., the region that expresses mainly D2 receptors). When the same authors tested the effects of chronic versus acute treatment with quinpirole, a high affinity D2/D3 receptor agonist, they found increased CRP40 levels in the nucleus accumbens shell region (e.g., the region that expresses D1 receptors) following chronic treatment only (Gabriele et al., 2007). Again, the results suggested that modulation by haloperidol and quinpirole was specific to the CRP40 protein, with no effects observed on Hsp70 (Gabriele et al., 2007).

Localization studies on the human form of CRP40 were recently performed by Gabriele and colleagues (2009). The authors reported cytoplasmic localization of CRP40 in the ventral striatum of healthy post-mortem as well as in drug naïve Schizophrenic patients' brain samples (Gabriele et al., 2009). In contrast, the localization of CRP40 shifted to the nucleus in post-mortem brain samples of haloperidol-treated Schizophrenia patients, which is consistent with mortalin function following cell stress (Gabriele et al., 2009). In this instance, it is hypothesized that the shifted localization of CRP40 to the nucleus may have been due to oxidative stress induced by increased DA concentrations resulting from haloperidol treatment (Gabriele et al. 2009). The specific role of CRP40 in the nucleus remains to be discovered, however, bioinformatic analysis suggests that CRP40 may possess a leucine zipper motif, which may serve as an activator of transcription (Gabriele et al., 2009).

CRP40 is downregulated in post-mortem brain specimens of PD patients and in blood platelets of PD patients. In 2005, Gabriele and colleagues found that CRP40 levels were significantly reduced in the brains of schizophrenic patients compared to the healthy control group. Un-medicated Schizophrenia patients showed the lowest levels of CRP40. CRP40 levels in brain samples of subjects treated with clozapine or haloperidol were slightly higher than those of un-medicated patients, however the levels were still reduced in comparison to normal, healthy controls. CRP40 has now been identified as a potential therapeutic and diagnostic molecule for certain dopamine-driven neurological disorders, including Parkinson's disease and schizophrenia (WO/2007/071045 filed Dec. 21, 2006).

Several studies have found that CRP40 is expressed solely in the CNS and blood, unlike mortalin which is found ubiquitously (Gabriele et al., 2009; Ross et al., 1995). As well, the CRP40 sequence is identical to the C-terminal region of mortalin and lacks the p53-binding domain which is located on the N-terminus of mortalin (Gabriele et al., 2009). Since CRP40 does not seem to possess the functional properties of mortalin, which regulates cell proliferation, it is possible to consider CRP40 as a potential therapeutic agent in neurodegenerative diseases. One disadvantage of investigating CRP40 as a possible therapeutic agent for neurological diseases is its size (40 kDa).

It is desirable to identify CRP40 fragments that have the same beneficial functions as full length CRP40 with reduced size. It was initially thought that the beneficial effect of CRP40 in neurological disorders was attributable at least in part to its dopamine-binding function.

### SUMMARY

In general, the present disclosure relates to novel fragments of human CRP40 and polynucleotides encoding them.

In one aspect, there are provided functional fragments of CRP40 comprising at least a portion of a putative substrate binding region of CRP40 that is not involved in dopamine binding.

In one embodiment, there is provided an isolated human CRP40 polypeptide fragment having a molecular weight of less than 30 kDa and comprising at least a functional portion of P2P4 (SEQ ID NO:5), or a functionally equivalent variant, fragment or derivative thereof. In some embodiments, the polypeptide has a molecular weight is less than 29, 28, 27, 26, 25, 24, 23, 22, 21, 20, 19, 18, 17, 16, 15, 14, 13, 12, 11, 10, 9, 8, 7, 6, 5, 4, 3, 2 or 1 kDa. In some embodiments, the polypeptide comprises at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 37, 33, 34, 35, 36, 37, 38, 39, 40, 41, 42, 43, 44, 45, 46, 47, 48, 49, 50, 51, 52, 53, 54, 55, 56, 57, 58, 59 or 60 contiguous amino acids of P2P4 (SEQ ID NO: 5). In some embodiments, the polypeptide comprises at least 10 contiguous amino acids of P2P4 (SEQ ID NO: 5). In some embodiments, the polypeptide comprises P1P4 (SEQ ID NO: 3), P1P5 (SEQ ID NO: 4), P2P4 (SEQ IDNO: 5) or P2P5 (SEQ ID NO: 6). In some embodiments, the polypeptide comprises P2P4 (SEQ ID NO: 5). In some embodiments, the polypeptide comprises a P2P4 fragment having an amino acid sequence selected from the group consisting of SEQ ID NOs: 9, 10, 11, 12, 13, 14, 15, 16, 17, 18, 19, 20, 21 and 22.

In some embodiments, the polypeptide is a functional variant having a sequence identity of at least 80%, 85%, 90%, 91%, 92%, 93%, 94%, 95%, 96%, 97%, 98% or 99%. In some embodiments, the functional variant has a sequence identity of at least 98%. In some embodiments, the functional variant comprises 1, 2, 3, 4, or 5 conservative modifications.

In some embodiments, the polypeptide consists of P2P4 (SEQ ID NO: 5).

In some embodiments, the functional portion of the polypeptide comprises all or part of a substrate binding region of CRP40. In some embodiments, the substrate binding region comprises a phosphorylation site for PKC, PKA and/or CK1.

In some embodiments, the polypeptide inhibits rotation in a 6-OHDA model by at least 25% (or 50% or 80%) compared to control when assessed at Day 4 post-administration.

In some embodiments, the polypeptide does not bind dopamine. For example, the polypeptide may lack a dopamine binding motif.

In some embodiments, there is provided a polypeptide encoded by a nucleic acid molecule having the nucleic acid sequence set forth in SEQ ID NO: 8; or a polynucleotide sequence with at least 80% sequence identity to SEQ ID NO:8 which hybridizes to the complement of SEQ ID NO:8 under stringent conditions. In some embodiments, the polypeptide is encoded by a nucleic acid molecule having the nucleic acid sequence set forth in SEQ ID NO: 8.

In another aspect, there is provided and isolated nucleic acid molecule encoding a polypeptide fragment as defined herein. In some embodiments, the nucleic acid molecule selected from the group consisting of:

a) nucleic acid molecule comprising at least 15, 25, 30, 60, 75, 90, 105, 120, 135, 150, 165, or 180 contiguous nucleotides of the sequence set forth in any one of SEQ ID NOs: 8, 51, 52 and 53;

b) a fragment, variant or derivative of a) having at least at least 80%, 85%, 90%, 95%, 96%, 97%, 98% or 99% sequence identity thereto;

c) a nucleic acid molecule that hybridizes to the complement of the nucleic acid molecule of a) or b) under moderately stringent conditions; and

d) a nucleic acid molecule of a), b) or c) which encodes a functional CRP40 fragment.

In some embodiments, the moderately stringent conditions comprise hybridization in 6× sodium chloride/sodium citrate (SSC) at 45° C., followed by one or more washes in 0.2×SSC, 0.1% SDS at 50-65° C.

In some embodiments, there is provided an isolated nucleic acid molecule comprising at least 60 contiguous nucleotides of the sequence set forth in any one of SEQ ID NOs: 8, 51, 52 and 53 or a variant thereof having at least 80% sequence identity thereto and encoding a functional CRP40 fragment. In some embodiments, the isolated nucleic acid molecule comprises the sequence set forth in any one of SEQ ID NOs: 8, 51, 52 and 53. In some embodiments, the isolated nucleic acid molecule comprises at least 30 contiguous nucleotides of the sequence set forth in SEQ ID NO: 8. In some embodiments, the isolated nucleic acid molecule comprises a sequence as set forth in any one of SEQ ID NO: 32-45. In some embodiments, the isolated nucleic acid molecule comprises the sequence set forth in SEQ ID NO: 8.

In another aspect, there is provided a vector comprising an isolated nucleic acid molecule as described herein. In one embodiment, the vector is a pGEX-2T vector.

In another aspect, there is provided a (host) cell comprising the vector described above. In one embodiment, the cell is a SHSY-5Y cell.

In another aspect, there is provided a primer comprising a polynucleotide consisting of at least 18 contiguous nucleotides of the nucleotide sequence of any one of SEQ ID NOS: 23-31 useful for preparing a CRP40 fragment. In some embodiments, the primer comprises a polynucleotide comprising the nucleotide sequence of any one of SEQ ID NOS: 23-31 useful for preparing a CRP40 fragment. In some embodiments, the primer consists of a polynucleotide having the nucleotide sequence of any one of SEQ ID NOS: 23-31 useful for preparing a CRP40 fragment.

In another aspect, there is provided a CRP40 polynucleotide prepared from any of the following primer pairs: a) B1F and ESR; b) B1F and E4R; c) B2F and ESR; d) B2F and E4R; e) B3F and E4R; or f) B3F and ESR.

In another aspect, there is provided a method of treating a neurological disorder in a subject in need thereof comprising administering to the subject a therapeutically effective amount of a CRP40 polypeptide fragment as defined herein or a polynucleotide encoding a CRP40 polypeptide fragment as define herein. In some embodiments, the method comprises administering to the subject a therapeutically effective amount of a CRP40 polypeptide fragment as defined herein. In some embodiments, the method comprises administering to the subject a therapeutically effective amount of polynucleotide encoding a CRP40 polypeptide fragment as define herein, e.g., in a vector capable of expressing said polynucleotide.

In another aspect, there is provided a use of the CRP40 fragment as defined herein or a polynucleotide encoding a CRP fragment as defined herein for the treatment of a neurological disorder. In another aspect there is provided, a use of the CRP40 fragment as defined herein or a polynucleotide encoding a CRP fragment as defined herein in the manufacture of a medicament for the treatment of a neurological disorder. In another aspect there is provided, a CRP40 fragment as defined herein or a polynucleotide encoding a CRP fragment as defined herein for use in the treatment of a neurological disorder. In another aspect there is provided, a CRP40 fragment as defined herein or a polynucleotide encoding a CRP fragment as defined herein for use in the manufacture of a medicament for the treatment of a neurological disorder. The neurological disorder is as described above.

In some embodiments of the method or use, the neurological disorder is characterized by one or more of: (a) oxidative stress, mitochondrial dysfunction and/or abnormal protein folding; (a) dopamine dysregulation; and (c) movement impairment. In some embodiments, the neurological disorder is characterized by one or more of oxidative stress, mitochondrial dysfunction and/or abnormal protein folding. In some embodiments, the neurological disorder is characterized by dopamine dysregulation. In some embodiments, the neurological disorder is characterized by movement impairment.

In some embodiments described herein, the neurological disorder is Parkinson's, a Parkinson-related disorder, tardive dyskinesia, drug-induced dyskinesia, cerebral ischemia, schizophrenia, bipolar disorder, an autistic disorder, Alzheimer's, Huntington's, ALS, ataxia telangiectasia, brain damage, dementia, diabetic neuropathy, dyslexia, dystonia, fetal alcohol syndrome, stroke, mini-stroke (transient ischemic attack), neurological sequelae of lupus, Neimann-Pick disease, Rett syndrome, sensory processing disorder, Tay-Sacs disease, Tourette syndrome, traumatic brain injury, Wilson's disease, Down's syndrome, multiple sclerosis, amyotrophic lateral sclerosis, hypoxia, ADHD or depression.

In some embodiments, the neurological disorder is Parkinson's disease, a Parkinson-related disorder, tardive dyskinesia or drug-induced dyskinesia. In some embodiments, the neurological disease is Parkinson's disease. In some embodiments, the neurological disorder a Parkinson-related disorder, such as, Lewy-body dementia or multiple systems atrophy. In some embodiments, the neurological disorder is tardive dyskenisia or drug-induced dyskinesia. In some embodiments, the drug-induced dyskinesia is L-dopa-induced or neuroleptic-induced.

In another aspect, there is provided a pharmaceutical composition comprising the polypeptide fragment as described herein or a polynucleotide encoding a CRP fragment as defined herein a pharmaceutically acceptable diluent or carrier. In some embodiments, the polynucleotide may be encompassed within a vector.

In another aspect, there is provided an antibody against a CRP40 fragment as defined herein.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1 is a diagram showing the sequences of exemplary fragments (P1P3, P1P4, P1P5, P2P4, P2P5 and a 48 AA peptide piece) of CRP40 and their alignment to full length CRP40.

FIG. 2 is a schematic outlining the stereotaxic surgery protocols for the 6-hydroxydopamine (6-OHDA) rat model.

FIG. 3 is a diagram showing the rotational behavior of animals treated with fragments P1P5, P1P4, P2P4, and a 48 AA peptide piece compared to control in the preclinical 6-OHDA rat model.

FIG. 4 is a summary of a bioinformatic analysis of P2P4 showing the sequences of 14 smaller fragments of P2P4 and their sequence alignment with DnaK.

FIG. 5 shows the polynucleotide and amino acid sequences of exemplary CRP40 fragments disclosed herein.

FIG. 6 is a graph showing that overexpression of CRP40 and mortalin partially preserves mitochondrial homeostasis and ROS levels under conditions of oxidative stress induced by treatment with H₂O₂ (500 μM).

FIG. 7 is a graph showing that overexpression of CRP40 and mortalin partially preserve ATP levels and cell viability in SH-SY5Y cells subjected to 16 hours of 5 μM MG-132 proteasomal inhibitor.

FIG. 8 shows comparison of specific binding of [3H]-DA by CRP40 protein in competition with cold DA, to specific binding of [3H]-DA by P2P4 fragment in competition with cold DA, and HSP47 control that does not bind dopamine. The results indicate that unlike CRP40 which binds dopamine with high capacity and low affinity, P2P4 does not bind dopamine at all.

FIG. 9 shows specific binding of [3H]-DA by CRP40, P2P4, and HSP47 proteins in competition with cold apomorphine, and indicates that HSP47 negative control and P2P4 do not bind apomorphine, a dopamine agonist, in comparison to CRP40, which binds dopamine.

FIG. 10 illustrates some of the putative functional characteristics of CRP40.

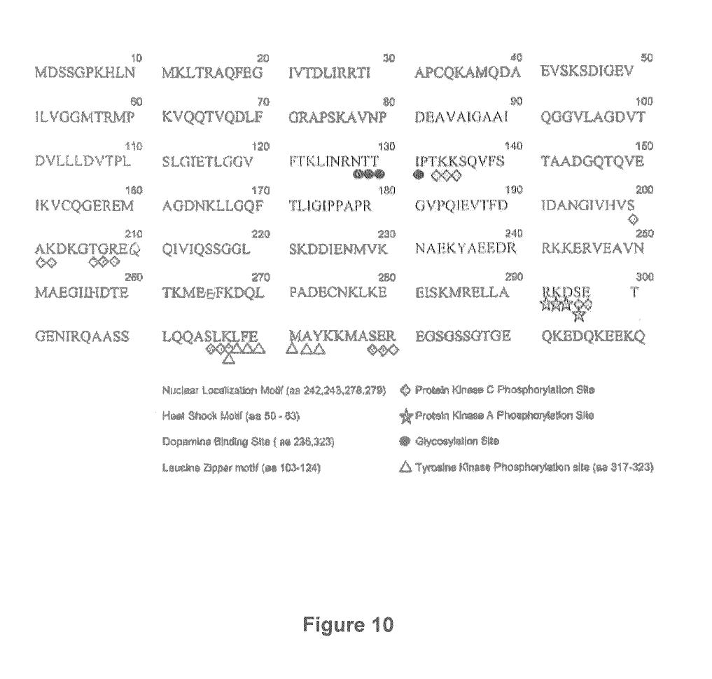

FIG. 11 illustrates a hypothetical 3D model of CRP40 showing putative functional domains and binding sites.

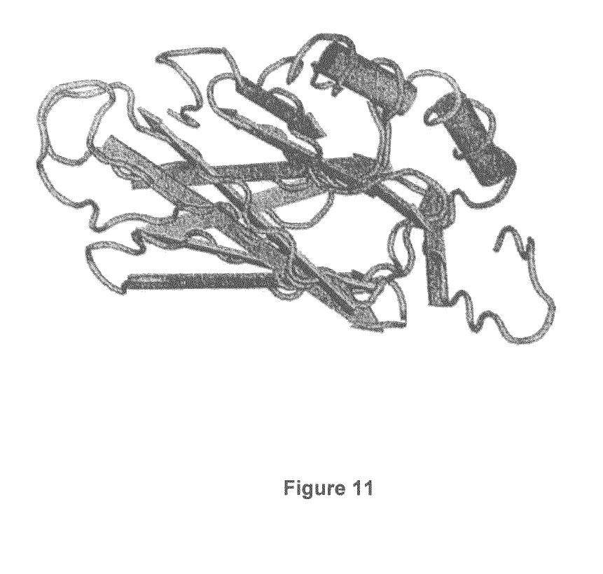

FIG. 12. Is a scheme outlining some predicted CRP40 protein binding interactions.

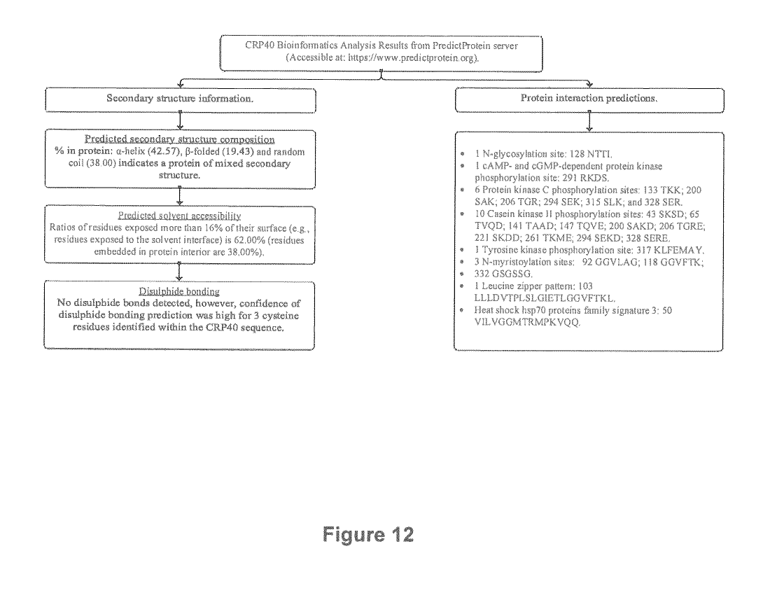

### DETAILED DESCRIPTION

The present disclosure relates to novel fragments of human CRP40 protein and polynucleotides encoding them. More particularly, the present disclosure relates to novel fragments of CRP40 comprising at least a functional portion of a putative substrate binding region that is not involved in dopamine binding.

The present inventors recently found that CRP40 could correct movement impairments in a preclinical model of PD and dyskinesia, the 6-hydroxydopamine (6-OHDA) model. It was thought that the effect of CRP40 was likely attributable to its known dopamine-binding function. It has now surprisingly been demonstrated that CRP40 fragments lacking dopamine binding function were as effective as full-length CRP40 in the 6-OHDA model.

PD is degenerative neurological disease characterized by oxidative stress, mitochondrial dysfunction, and protein misfolding. Given that CRP40 belongs to a family of molecular chaperone proteins, the present inventors postulate that CRP40 plays a neuroprotective role as a multifunctional molecular chaperone protein that ensures correct protein folding and cell stability. In addition, it is hypothesized that CRP40 may protect mitochondria and neurons against oxidative stress. It is therefore predicted that the CRP40 fragments disclosed herein may be useful in the treatment of Parkinson's disease and other neurological disorders characterized by one or more of oxidative stress, mitochondrial dysfunction and abnormal protein folding.

Several fragments of CRP40 were made and tested. SEQ ID NO:1 refers to the amino acid sequence of the full-length CRP40 protein (40 k Da). SEQ ID NO:2 refers to the amino acid sequence of the P1P3 fragment of human CRP40 (11.1 kDa). SEQ ID NO:3 refers to the amino acid sequence of the P1P4 fragment of human CRP40 (16.6 kDa). SEQ ID NO:4 refers to the amino acid sequence of the P1P5 fragment of human CRP40 (27 kDa). SEQ ID NO:5 refers to the amino acid sequence of the P2P4 fragment of human CRP40 (6.6 kDa); SEQ ID NO:6 refers to the amino acid sequence of the P2P5 fragment of human CRP40 (9.26 kDa). SEQ ID NO:7 refers to the amino acid sequence of a synthesized peptide piece (48 aa). SEQ ID NO:8 refers to the nucleotide sequence encoding the full length P2P4 peptide.

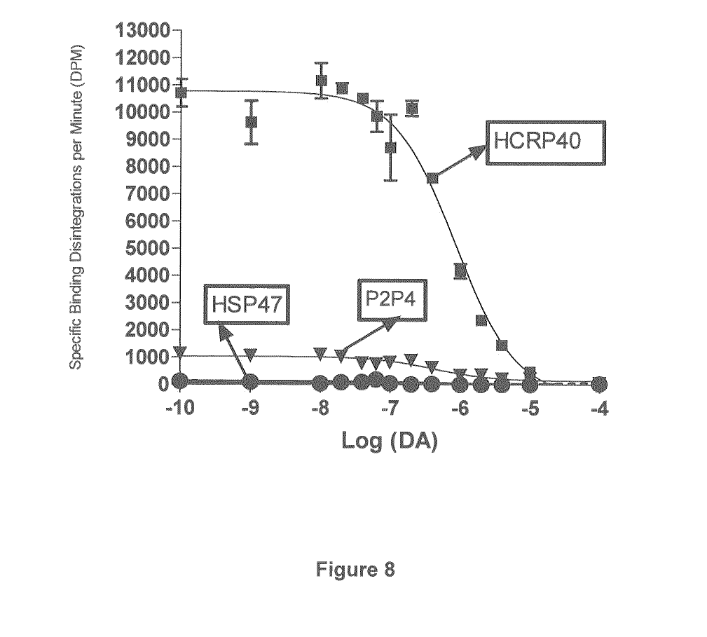

P2P4 is a 60-amino acid fragment spanning amino acids 95-154 of the full-length CRP40 protein (SEQ ID NO:1). It was the smallest functional fragment of the fragments recited above. The P2P4 fragment was found to be as effective as full-length CRP40 in the 6-OHDA rat model of dyskinesia, a well-established animal model of Parkinson's disease. It was previously thought that the beneficial effect of CRP40 was due at least in part to its effect on dopamine. However, protein binding studies confirmed that P2P4 does not bind dopamine (FIG. 8). It was unexpected that CRP40 fragments lacking dopamine-binding function would be functional in the 6-OHDA model. Based on homology with other members of the Hsp70 family, e.g. DnaK, P2P4 is now believed to contain a putative substrate binding region that is important for CRP40 function. Hypothetically, P2P4 is in the putative region of a leucine zipper motif where this region may act as a transcriptional factor. Two larger CRP40 fragments comprising P2P4 (P1P4, P1P5) were also effective in the 6-OHDA model whereas fragments that did not include this region (P1P3 and a 48-amino acid peptide piece) were not effective. Although P2P5 was not tested in the 6-OHDA model, it is expected to be functional since it contains the functional P2P4 fragment. This is an important finding since, when investigating potential new therapeutic molecules, smaller functional fragments are preferred over full-length proteins. Advantageously, the P2P4 fragment (6.6. kDa) is 6 times smaller than the full-length CRP40 protein (40 kDa).

One aspect of the present disclosure relates to an isolated human CRP40 polypeptide fragment having a molecular weight of less than 30 kDa and comprising at least a functional portion of P2P4 (SEQ ID NO:5), or a functionally equivalent variant, fragment or derivative thereof.

In some embodiments, the CRP40 fragment has a molecular weight of less than 29, 28, 27, 26, 25, 24, 23, 22, 21, 20, 19, 18, 17, 16, 15, 14, 13, 12, 11, 10, 9, 8, 7, 6, 5, 4, 3, 2 or 1 kDa. In some embodiments, the CRP40 fragment has a molecular weight of less than 10 kDa. In some embodiments, the molecular weight is less than 7 kDa. In some embodiments, the molecular weight is less than 5 kDa. In some embodiments, the molecular weight is less than 1 kDa.

In some embodiments, the CRP40 fragment comprises at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 37, 33, 34, 35, 36, 37, 38, 39, 40, 41, 42, 43, 44, 45, 46, 47, 48, 49, 50, 51, 52, 53, 54, 55, 56, 57, 58, 59 or 60 contiguous amino acids of P2P4 (SEQ ID NO: 5). In one embodiment, the CRP40 fragment comprises at least 5 contiguous amino acids of P2P4 (SEQ ID NO: 5). In one embodiment, the CRP40 fragment comprises at least 10 contiguous amino acids of P2P4 (SEQ ID NO: 5).

In some embodiments, the CRP40 fragment comprises between about 5-10, 10-20, 20-30, 30-40, 40-50 or 50-60 contiguous amino acids of P2P4 (SEQ ID NO: 5). In a particular embodiment, the CRP40 fragment comprises 30-60 contiguous amino acids of P2P4 (SEQ ID NO: 5).

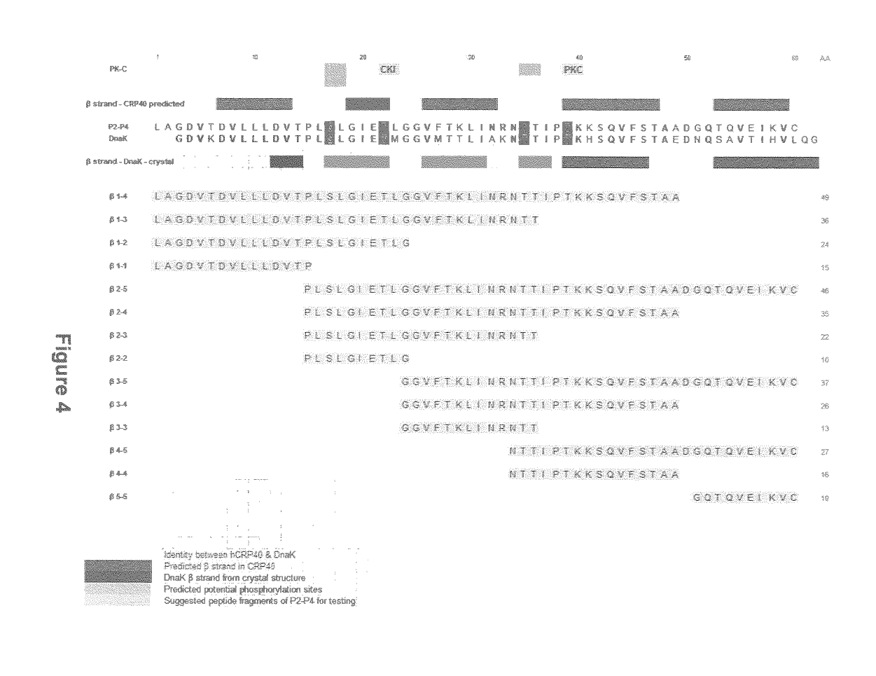

It is believed that fragments smaller than the P2P4 (60 aa) fragment will be functional. FIG. 4 shows 14 smaller fragments of P2P4 and their sequence alignment with DnaK. The fragments span various portions of the 5 predicted β-sheets within the putative substrate binding region of CRP40. SEQ ID NO: 9 refers to the amino acid sequence of the β1-4 fragment of human CRP40 (49 aa); SEQ ID NO:10 refers to the amino acid sequence of the β1-3 fragment of human CRP40 (36 aa); SEQ ID NO:11 refers to the amino acid sequence of the β1-2 fragment of human CRP40 (24 aa); SEQ ID NO:12 refers to the amino acid sequence of the β1-1 fragment of human CRP40 (15 aa); SEQ ID NO:13 refers to the amino acid sequence of the β2-5 fragment of human CRP40 (46 aa); SEQ ID NO:14 refers to the amino acid sequence of the β2-4 fragment of human CRP40 (35 aa); SEQ ID NO:15 refers to the amino acid sequence of the β2-3 fragment of human CRP40 (22 aa); SEQ ID NO:16 refers to the amino acid sequence of the β2-2 fragment of human CRP40 (10 aa); SEQ ID NO:17 refers to the amino acid sequence of the β3-5 fragment of human CRP40 (37 aa); SEQ ID NO:18 refers to the amino acid sequence of the β3-4 fragment of human CRP40 (26 aa); SEQ ID NO:19 refers to the amino acid sequence of the β3-3 fragment of human CRP40 (13 aa); SEQ ID NO:20 refers to the amino acid sequence of the β4-5 fragment of human CRP40 (27 aa); SEQ ID NO:21 refers to the amino acid sequence of the β4-4 fragment of human CRP40 (16 aa); and SEQ ID NO:22 refers to the amino acid sequence of the β5-5 fragment of human CRP40 (10 aa). The polynucleotide sequences encoding the smaller P2P4 fragments are provided in SEQ ID: NOs 32-45.

In some embodiments, the polypeptide fragment is less than 250, 200, 100, 90, 80, 70, 60, 50, 40, 30, 20, 10, 5, or 3 amino acids in length. In some embodiments the the polypeptide fragment is between 10-250, 10-150, 10-100, or 10-60 amino acids in length. In some embodiments the the polypeptide fragment is between 5-60, 10-60, 20-60, 30-60, 40-60 or 50-60 amino acids in length.

The term “isolated” as used herein means essentially pure and free from extraneous cellular material. For example, isolated CRP40 fragments are polypeptides that are essentially pure and free from extraneous cellular material including other proteins or polypeptide fragments. For example, isolated nucleic acid molecule refers to a polynucleotide sequence that is essentially pure and free from extraneous cellular material.

The term “polypeptide” as used herein refers to a sequence of naturally occurring and/or artificial amino acids covalently linked via peptide bonds. A polypeptide includes a polypeptide that has been isolated from a naturally occurring source, a polypeptide that has been synthetically produced, or produced using recombinant techniques. It is to be appreciated that a polypeptide that includes a leader or pro-sequence, a tag, a label, a signal peptide, or a polypeptide that undergoes a post translational modification, is intended to fall within the definition of a polypeptide fragment.

The term “fragment” as used herein refers to a portion of a larger reference molecule. For example, a fragment of human CRP40 protein refers to a polypeptide having a partial amino acid sequence as compared to the full-length sequence of CRP40 (SEQ ID NO: 1).

A “functional portion” of a polypeptide fragment refers to a portion of the molecule encompassing a region, domain or motif having a particular function. A functional portion may comprise one or more regions, motifs or domains that are associated with a particular activity. For example, although the exact functionality of P2P4 has not yet been elucidated, it is known that a portion of the sequence within the P2P4 fragment is important for CRP40 function. Looking at the alignment pattern between P2P4 and DnaK, the P2P4 fragment is thought to contain a putative substrate binding region of CRP40 containing 5 predicted B-strands. Based on homology with DnaK, some of the functionality within the substrate binding region may be attributable to phosphorylation sites within this region, e.g. the P2P4 contains putative phosphorylation sites for PKC, CKI or PKA. In some embodiments, the functional portion of P2P4 comprises all or part of a substrate binding region of CRP40. In some embodiments, the substrate binding region comprises a phosphorylation site for protein kinase A (PKA), protein kinase C (PKC), and/or casein kinase 1 (CK1). P2P4 is hypothesized to be within the putative leucine zipper motif of CRP40.

The term “functionally equivalent variant” is intented to mean a polynucleotide or polypeptide sequence that has been modified by substitution, insertion or deletion of one or more (e.g. 1, 2, 3, 4, 5, 6, 7, 8, 9 10) nucleotides or one or more (e.g. 1, 2, 3, 4, 5, 6, 7, 8, 9, 10) amino acids, but that has substantially the same or better activity as the reference sequence. Function of a polypeptide may be assessed experimentally, for example, by determining activity in an in vitro or in vivo experiment. Whether or not a given polypeptide or polynucleotide is “functional” may be determined by first selecting an appropriate function to asses. In some cases, a functional molecule may be one that exhibits the desired function to a statistically significant degree (e.g. p<0.05; <0.01; <0.001).

In some cases, the function of a CRP40 fragment or functionally equivalent variant thereof may be assessed in vitro, e.g. ability to protect cells from oxidative stress. In some cases, the function of a CRP40 fragment or functionally equivalent variant thereof may be assessed in vivo, e.g. ability to inhibit rotation in the 6-OHDA model. In the case of a polynucleotide, function may be determined by its ability to encode a functional CRP40 fragment. A skilled person will be able to select a suitable parameter to assess function of a given polypeptide or polynucleotide.

In some embodiments, a CRP40 fragment or functionally equivalent variant thereof, or a polynucleotide encoding same, is capable of inhibiting rotation in the 6-OHDA model to a statistically significant degree (e.g. p<0.05; <0.01; <0.001) compared to control (e.g. when measured at Day 4-12, Day 4-8, Day 4, Day 8). In some embodiments, a CRP40 fragment or functionally equivalent variant thereof, or a polynucleotide encoding same, is capable of inhibiting rotation in the 6-OHDA model by at least 10, 20, 25, 30, 40, 50, 60, 70, 80, 90% compared to control (e.g. when measured at Day 4-12, Day 4-8, Day 4, Day 8). In some embodiments, the measurement is taken at Day 4 post-administration. In some embodiments, the measurement is taken at Day 8 post-administration. In some embodiments, the polypeptide inhibits rotation in a 6-OHDA model by at least 25% compared to control when assessed at Day 4 post-administration. In some embodiments, the polypeptide inhibits rotation in a 6-OHDA model by at least 50% compared to control when assessed at Day 4 post-administration. In some embodiments, the polypeptide inhibits rotation in a 6-OHDA model by at least 80% compared to control when assessed at Day 4 post-administration.

A variant may contain one or more conservative amino acid substitutions. A “conservative substitution” is one in which an amino acid is substituted for another amino acid that has similar properties, such that one skilled in the art of peptide chemistry would expect the secondary structure and properties of the polypeptide to be substantially unchanged. As is known in the art, the twenty naturally occurring amino acids can be grouped according to the physicochemical properties of their side chains. Suitable groupings include alanine, valine, leucine, isoleucine, proline, methionine, phenylalanine and tryptophan (hydrophobic side chains); glycine, serine, threonine, cysteine, tyrosine, asparagine, and glutamine (polar, uncharged side chains); aspartic acid and glutamic acid (acidic side chains) and lysine, arginine and histidine (basic side chains). Another grouping of amino acids is phenylalanine, tryptophan, and tyrosine (aromatic side chains). A conservative substitution involves the substitution of an amino acid with another amino acid from the same group, while a non-conservative substitution involves the substitution of an amino acid with another amino acid from a different group.

In some cases, it may be desirable to substitute one or more specific amino acids within a particular region of interest, for example, to activate or deactivate a binding site within that region. For example, if a particular CRP40 fragment is found to bind p53, it may be desirable to mutate the p53 binding site for therapeutic applications. Losefson & Azem (2010) showed that p53 binds mortalin in the peptide binding region of the protein. They were able to interfere with p53 binding by introducing a single point mutation (mutant V482F). A similar approach could be used for CRP40 fragments determined to bind p53. Well-known techniques such as site-directed mutagenesis may be used.

The “sequence identity” is determined by comparing two optimally aligned sequences over a comparison window, wherein the portion of the nucleic acid or amino acid sequence in the comparison window may comprise additions, deletions (i.e., gaps), or substitutions as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. The sequence identity can be any integer from about 80% to 100%. Typically, a variant sequence comprises at least about 80% (e.g. 80%, 81%, 82%, 83%, 84%, 85%, 86%, 87%, 88%, 89%, 90%, 91%, 92%, 93%, 94%, 95%, 96%, 97%, 98%, 89.5%, 99% or 99.5%) sequence identity compared to a reference sequence using available programs using standard parameters. The person skilled in the art will understand that optimal alignment of sequences for comparison may be conducted by the local homology algorithm, by the homology alignment algorithm, by the search for similarity method, by computerized implementations of these algorithms (GAP, BESTFIT, BLAST, FASTA, TFASTA, and DASH), or by inspection. In some embodiments, the sequence identity is at least 90%. In some embodiments, the sequence identity is at least 95%. In some embodiments, the sequence identity is at least 99%. The comparison window may be the entire length of a polypeptide or polynucleotide or it may be a particular segment of a polypeptide or polynucleotide. For example, the comparison window may be a segment comprising a functional portion of a CRP40 fragment (e.g. polypeptide), such as P2P4, or a segment encoding a functional portion of a CRP40 fragment (e.g. polynucleotide).

The term “derivative” as used herein refers to amino acid sequence that has been altered in some way to produce a polypeptide having a desired characteristic, such as, increased stability. For example, amino acids can be replaced by the same amino acid of different chirality, or non-naturally occurring amino acids can be inserted or substituted in the polypeptide. Alternatively, the polypeptide may be chemically modified, e.g. to improve pharmacokinetics, such as by crosslinking with polymers such as polyethylene glycol. Such derivatives may have increased serum half lives in vivo, bioavailability, dissociation rates and other properties that make them useful in formulating pharmaceutical compositions.

The polypeptides disclosed herein can also be produced as fusion proteins. The term “fusion protein” refers to a chimeric protein containing the polypeptide of interest (i.e., a CRP40 fragment) joined to an exogenous protein fragment. The fusion partner may, for example, provide a detectable moiety, may provide an affinity tag to allow purification of the recombinant fusion protein from the host cell, may provide a targeting moiety, may provide an an additionally enzymatic activity, and the like. If desired, the fusion partner may be removed from the protein of interest by a variety of enzymatic or chemical means known to the art. One use of such fusion proteins is to improve the purification or detection of the polypeptide or peptide. For example, a polypeptide or peptide can be fused to an immunoglobulin Fc domain and the resultant fusion protein can be readily purified using a protein A column. Other examples of fusion proteins include polypeptides fused to histidine tags (allowing for purification on Nie+ resin columns), to glutathione-S-transferase (allowing purification on glutathione columns) or to biotin (allowing purification on streptavidin columns or with streptavidin labelled—19 magnetic beads). Once the fusion protein has been purified, the tag may be removed by site-specific cleavage using chemical or enzymatic methods known in the art.

The present disclosure also provides polynucleotides encoding the polypeptides described above. The term “polynucleotide” or “nucleic acid molecule” refers to a sequence of several nucleotides. As used herein the term “encodes” refers to a polynucleotide which comprises the information for translation into a specified polypeptide, such as a CRP40 polypeptide fragment. The term polynucleotide having a nucleic acid sequence encoding a specified polypeptide refers to a nucleic acid sequence comprising the coding region of a gene, a gene product or a portion of a gene product. The coding region may be present in a cDNA, genomic DNA, or RNA form. When present in a DNA form, the ploynucleotide may be single-stranded (i.e., the sense strand) or double-stranded. Suitable control elements such as enhancers, promoters, splice junctions, polyadenylation signals, etc., may be placed in close proximity to the coding region if needed to permit proper initiation of transcription and/or correct processing of the primary RNA transcript. Alternatively, the coding region utilized in the expression vectors of the present invention may contain endogenous enhancers, exogenous promoters, splice junctions, intervening sequences, polyadenylation signals, etc., or a combination of both endogenous and exogenous control elements.

In some embodiments, the isolated nucleic acid molecule is a nucleic acid molecule comprising at least 15, 25, 30, 60, 75, 90, 105, 120, 135, 150, 165, or 180 contiguous nucleotides of the sequence set forth in any one of SEQ ID NOs: 8, 51, 52 and 53. In some embodiments, the nucleic acid molecule comprises a fragment, variant or derivative of a nucleic acid molecule comprising at least 15, 25, 30, 60, 75, 90, 105, 120, 135, 150, 165, or 180 contiguous nucleotides of the sequence set forth in any one of SEQ ID NOs: 8, 51, 52 and 53, which has at least at least 80%, 85%, 90%, 95%, 96%, 97%, 98% or 99% sequence identity to the reference sequence. A variant may for example contain 1-10 (e.g. 1, 2, 3, 4, 5, 6, 7, 8, 9, 10) substitutions, additions or deletions. In some cases, the variant comprises 1-10 substitutions. In some embodiments, conservative substitutions substitutions within the same group are preferred, e.g. A, T, U; or G, C.

In some embodiments, the isolated nucleic acid molecule comprises the sequence set forth in any one of SEQ ID NOs: 8, 51, 52 and 53. In some embodiments, the isolated nucleic acid molecule consists of the sequence set forth in any one of SEQ ID NOs: 8, 51, 52 and 53. In some embodiments, the isolated nucleic acid molecule comprises at least 30 (e.g. 30, 60, 75, 90, 105, 120, 135, 150, 165, or 180) contiguous nucleotides of the sequence set forth in SEQ ID NO: 8, which encodes the CRP40 fragment, P2P4 (SEQ ID NO: 5). In some embodiments, the isolated nucleic acid molecule comprises the sequence as set forth in any one of SEQ ID NO: 8. In some embodiments, the isolated nucleic acid molecule consists of the sequence as set forth in SEQ ID NO: 8.

In some embodiments, the isolated nucleic acid molecule encodes a P2P4 fragment, such as a fragment described in any one of SEQ ID NOs: SEQ ID NOs: 9-22. In some embodiments, the isolated nucleic acid molecule encoding the P2P4 fragment comprises a sequence as set forth in any one of SEQ ID NO: 32-45 (e.g. SEQ ID NO: 32, 33, 34, 35, 36, 37, 38, 39, 40, 41, 42, 43, 44 or 45).

In some embodiments, the isolated nucleic acid molecule comprises at least 60 contiguous nucleotides of the sequence set forth in any one of SEQ ID NOs: 8, 51, 52 and 53 or a variant thereof having at least 80% sequence identity thereto and encoding a functional CRP40 fragment.

In some embodiments, the isolated nucleic acid molecule is a nucleic acid molecule that hybridizes to the complement of the nucleic acid molecule describe above under moderately stringent conditions. In some embodiments, the isolated nucleic acid molecule is a nucleic acid molecule that hybridizes to the complement of the nucleic acid molecule describe above under stringent conditions.

The term “stringent” refers to refers to conditions under which a probe will hybridize to its target subsequence, typically in a complex mixture of nucleic acid molecules, with little or no binding to other sequences. Stringent conditions are sequence-dependent and will be different in different circumstances. Longer sequences hybridize specifically at higher temperatures. Generally, stringent conditions are selected to be about 5-10° C. lower than the thermal melting point (Tm) for the specific sequence at a defined ionic strength pH. The Tm is the temperature (under defined ionic strength, pH, and nucleic acid molecule concentration) at which 50% of the probes complementary to the target hybridize to the target sequence at equilibrium (as the target sequences are present in excess, at Tm, 50% of the probes are occupied at equilibrium). Stringent hybridization conditions will be those in which the salt concentration is less than about 1.0 M sodium ion, typically about 0.01 M to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent hybridization conditions may also be achieved with the addition of destabilizing agents such as formamide. For selective or specific hybridization, a positive signal is at least two times background, optionally 10 times background hybridization. Exemplary stringent hybridization conditions can be as follows: 50% formamide, 5×SSC, and 1% SDS, incubating at 42° C., or 5×SSC, 1% SDS, incubating at 65° C., with wash in 0.2×SSC, and 0.1% SDS at 55° C., 60° C., or 65° C. Such washes can be performed for 5, 15, 30, 60, 120, or more minutes. The high stringency conditions used in these techniques are well known to those skilled in the art of molecular biology, and examples of them can be found, for example, in Ausubel et al., Current Protocols in Molecular Biology, John Wiley & Sons, New York, N.Y., 1998, and in Maniatis et al., in Molecular Cloning: A Laboratory Manual, Cold Spring Harbor Laboratory, (1982). Moderately stringent conditions may comprise hybridization in 6× sodium chloride/sodium citrate (SSC) at 45° C., followed by one or more washes in 0.2×SSC, 0.1% SDS at 50-65° C.

In some embodiments, the isolated nucleic acid molecules described herein encode functional CRP40 fragments. In particular, nucleic acid molecules for use in therapeutic applications encode functional CRP40 fragments. Thus, CRP40 fragments for use in therapeutic application are functional CRP40 fragments.

Using knowledge of the genetic code in combination with the amino acid sequences set forth herein, sets of degenerate polynucleotides (e.g. oligonucleotides) can be prepared. Such polynucleotides may be useful as primers, e.g., in polymerase chain reactions (PCR), whereby DNA fragments are isolated and amplified. In some embodiments, polynucleotides of the invention may be useful as probes or primers.

As used herein, “primer” refers to an oligonucleotide containing at least 10 contiguous nucleotides from which synthesis of a primer extension product can be initiated. Experimental conditions conducive to synthesis include the presence of nucleoside triphosphates and an agent for polymerization and extension, such as DNA polymerase, and a suitable buffer, temperature and pH. Primers typically comprise at least about 18 contiguous nucleotides of a DNA sequence, but may include up to 30, 40, 50 or 60 or more nucleotides.

In some embodiments, there is provided a primer comprising a polynucleotide consisting at least 18 contiguous nucleotides of the nucleotide sequence of any one of SEQ ID NOS: 23-31. In some embodiments, a primer is provided that comprises the nucleotide sequence of any one of SEQ ID NOS: 23-31. In some embodiments, a primer is provided that consists of the nucleotide sequence of any one of SEQ ID NOS: 23-31. In some embodiments, a CRP40 polynucleotide is prepared from one of the following primer pairs: a) B1F and E5R; b) B1F and E4R; c) B2F and E5R; d) B2F and E4R; e) B3F and E4R; or f) B3F and E5R.

The polynucleotides of the invention may be used to express CRP40 polypeptide fragments. Polynucleotides disclosed herein may be inserted into expression vectors, and operably linked to an expression control sequence, to generate constructs that are useful for producing CRP40 polypeptide fragment via heterologous expression. Suitable expression vectors for this purpose include but are not limited to: pGEX-2X, pET (e.g. pET30A), pMT2, Impact System or pMAL™ Protein Fusion and Purification System (available from New England Biolabs), pPICZα A, B, and C Pichia expression vectors for selection on Zeocin™ (available from Invitrogen), or the S30 T7 High-Yield Protein Expression System or TNT® SP6 High Yield Wheat Germ System (available from Promega). In one embodiment, the vector is pGEX-2X.

Suitable host cells for expression of the polypeptide include both eukaryotic and prokaryotic cells. In one embodiment, the host cells are SHSY-5Y cells. Mammalian host cells may also be employed, as may be insect cells. The polypeptide may also be produced in lower eukaryotes such as yeast or in prokaryotes such as bacteria. Suitable bacterial strains include, for example, Escherichia coli, Bacillus subtilis, Salmonella typhimurium, or any bacterial strain capable of expressing heterologous polypeptides. Potentially suitable yeast strains include Saccharomyces cerevisiae, Schizosaccharomyces pombe, Kluyveromyces strains, Candida, or any yeast strain capable of expressing heterologous polypeptides.

The polypeptides of the present invention can be prepared by methods known in the art, such as purification from cell extracts or the use of recombinant techniques. The term “recombinant” when made in reference to a nucleic acid molecule refers to a nucleic acid molecule that is comprised of segments of nucleic acid joined together by means of molecular biological techniques. The term “recombinant” when made in reference to a polypeptide refers to a polypeptide that is expressed using a recombinant nucleic acid molecule. Polypeptides as described herein will preferably involve purified or isolated polypeptide preparations. In certain embodiments, purification of the polypeptide may utilize recombinant expression methods well known in the art, and may involve the incorporation of an affinity tag into the expression construct to allow for affinity purification of the target polypeptide

The polypeptides of the present invention can be purified using standard techniques such as chromatography (e.g. ion exchange, affinity, and sizing column chromatography or high performance liquid chromatography), centrifugation, differential solubility, or by other techniques familiar to a worker skilled in the art. The polypeptides can also be produced by recombinant techniques. Typically this involves transformation (including transfection, transduction, or infection) of a suitable host cell with an expression vector comprising a polynucleotide encoding the protein or polypeptide.

Polypeptides of the invention may be prepared using heterologous expression techniques, e.g. by culturing a host cell that has been transformed with expression constructs comprising cDNA encoding a polypeptide of interest linked to expression control sequences, under culture conditions suitable to express the polypeptide of the invention. The resulting expressed polypeptide may then be purified from such culture using conventional purification processes, such as gel filtration and ion exchange chromatography. The purification of the polypeptide may also include an affinity column containing agents which will bind to the polypeptide; one or more column steps over such affinity resins as DEAE-sepharose, concanavalin A-agarose, heparin-toyopearl® or Cibacrom blue 3GA Sepharose®; one or more steps involving hydrophobic interaction chromatography using such resins as phenyl ether, phenyl sepharose, butyl ether, or propyl ether; or immunoaffinity chromatography. Alternatively, the polypeptide of the invention may also be expressed in a form that will facilitate purification. For example, it may be expressed as a fusion polypeptide, such as those of maltose binding polypeptide (MBP), glutathione-S-transferase (GST) or thioredoxin (TRX), or with a HIS tag. Kits for expression and purification of fusion polypeptides are commercially available from New England BioLab (Beverly, Mass.), Pharmacia (Piscataway, N.J.), and InVitrogen, respectively. The polypeptide can also be tagged with an epitope and subsequently purified by using a specific antibody directed to such epitope. One such epitope (“Flag”) is commercially available from Kodak (New Haven, Conn.). Finally, one or more reverse-phase high performance liquid chromatography (RP-HPLC) steps employing hydrophobic RP-HPLC media, e.g., silica gel having pendant methyl or other aliphatic groups, can be employed to further purify the polypeptide. Some or all of the foregoing purification steps, in various combinations, can also be employed to provide a substantially homogeneous recombinant polypeptide.

Shorter sequences can also be chemically synthesized by methods known in the art including, but not limited to, exclusive solid phase synthesis, partial solid phase synthesis, fragment condensation or classical solution synthesis. Methods for constructing the polypeptides of the invention by synthetic means are known to those skilled in the art. The synthetically-constructed polypeptide sequences, by virtue of sharing primary, secondary or tertiary structural and/or conformational characteristics with a native polypeptides may possess biological properties in common therewith, including functional activity.

Another aspect of the disclosure provides a method of treating a neurological disorder in a subject. The term “subject” or “patient” as used herein, refers to a mammal, preferably a human. Once prepared and suitably purified, the CRP40 fragments described herein, or the polynucleotides encoding them, may be useful in the treatment of various neurological disorders. For example, the CRP40 fragments described herein, or the polynucleotides encoding them, may be useful in the treatment of neurological disorders characterized by one or more of: (a) oxidative stress, mitochondrial dysfunction and/or abnormal protein folding; (b) dopamine dysregulation; and/or (c) movement impairment. As will be appreciated by those skilled in the art, some neurological disorders will fall into more than one of the above categories and may fall into all three. The term “disorder” as used herein is intended to capture diseases, disorders and conditions requiring treatment. As used herein “treatment” refers to prevention, reduction or amelioration of an unwanted symptom of a disorder.

In one aspect, there is provided a method of treating a neurological disorder in a subject in need thereof comprising administering to the subject a therapeutically effective amount of a CRP40 polypeptide fragment as defined herein, or a polynucleotide encoding a CRP40 polypeptide fragment as defined herein. In another aspect, there is provided a use of a CRP40 fragment as defined herein or a polynucleotide encoding a CRP fragment as defined herein for the treatment of a neurological disorder. In another aspect there is provided, a use of the CRP40 fragment as defined herein or a polynucleotide encoding a CRP fragment as defined herein in the manufacture of a medicament for the treatment of a neurological disorder. In another aspect there is provided, a CRP40 fragment as defined herein or a polynucleotide encoding a CRP fragment as defined herein for use in the treatment of a neurological disorder. In another aspect there is provided, a CRP40 fragment as defined herein or a polynucleotide encoding a CRP fragment as defined herein for use in the manufacture of a medicament for the treatment of a neurological disorder.

A skilled professional will be able to determine a “therapeutically effective amount” of a CRP40 fragment as defined herein or a polynucleotide encoding a CRP40 fragment as defined herein. In most cases, a low dose will be given initially and the dose will be gradually increased over time until a desired outcome is achieved (e.g. reduction of an unwanted symptom of a disorder), balancing efficacy against toxicity.

In some embodiments, the method or use comprises administering to the subject a therapeutically effective amount of a CRP40 polypeptide fragment as defined herein. In some embodiments, the method or use comprises administering to the subject a therapeutically effective amount of a polynucleotide encoding a CRP fragment as defined herein. The polynucleotide may, for example, be encompassed in a vector capable of expressing the polypeptide. The present inventors have previously demonstrated beneficial effects of administration of CRP40 protein or a vector expressing CRP40 protein (WO/2007/071045). Therefore, the present disclosure encompasses administration of CRP40 fragments as well as polynucleotides encoding said fragments (e.g. in a vector) since both are predicted to be effective.

In some embodiments, the neurological disorder is characterized by one or more of: (a) oxidative stress, mitochondrial dysfunction and/or abnormal protein folding; (a) dopamine dysregulation; and (c) movement impairment. In some embodiments, the neurological disorder is characterized by one or more of oxidative stress, mitochondrial dysfunction and/or abnormal protein folding. In some embodiments, the neurological disorder is characterized by oxidative stress. In some embodiments, the neurological disorder is characterized by mitochondrial dysfunction. In some embodiments, the neurological disorder is characterized by abnormal protein folding. In some embodiments, the neurological disorder is characterized by dopamine dysregulation. In some embodiments the neurological disorder is characterized by movement impairment.

In some embodiments, the neurological disorder is selected from the group consisting of Parkinson's, a Parkinson-related disorder, tardive dyskinesia, drug-induced dyskinesia, cerebral ischemia, schizophrenia, bipolar disorder, autism, Alzheimer's, Huntington's, ALS, ataxia telangiectasia, brain damage, dementia, diabetic neuropathy, dyslexia, dystonia, fetal alcohol syndrome, stroke, mini-stroke (transient ischemic attack), neurological sequelae of lupus, Neimann-Pick disease, Rett syndrome, sensory processing disorder, Tay-Sacs disease, Tourette syndrome, traumatic brain injury, Wilson's disease, Down's syndrome, hypoxia, ADHD or depression.

In some embodiments, the neurological disorder is Parkinson's disease, a Parkinson-related disorder, tardive dyskinesia or drug-induced dyskinesia. In some embodiments, the neurological disorder is Parkinson's disease. In some embodiments, the neurological disorder is a Parkinson-related disorder. A Parkinson-related disorder may include, but is not limited to, Lewy-body dementia or multiple systems atrophy.

In some embodiments, the neurological disorder is a dyskinesia, such as tardive dyskinesia or a drug-induced dyskinesia. Drug-induced dyskinesia may include, for example L-dopa-induced or neuroleptic-dyskinesia. Other drugs have also been known to cause Parkinson-like symptoms: neuroleptic antipsychotics especially the phenothiazines (such as perphenazine and chlorpromazine), thioxanthenes (such as flupenthixol and zuclopenthixol) and butyrophenones (such as haloperidol (Haldol)), piperazines (such as ziprasidone), and, rarely, antidepressants. Treatment of Parkinson-like symptoms associated with these medications is also encompassed.

In one particular embodiment, the method comprises administering to the patient a therapeutically effective amount of a human CRP40 fragment comprising the amino acid sequence as defined in SEQ ID NO: 5, or a functionally equivalent variant thereof exhibiting at least about 80% sequence homology with the amino acid sequence of SEQ ID NO:5, or a nucleic acid encoding said CRP40 fragment or functionally equivalent variant.

The method may comprise administering a vector comprising a nucleotide sequence that encodes a CRP40 fragment to a patient in need thereof. Suitable vectors and methods of cloning a nucleotide sequence would be known to a person of skill in the art. The vector may further comprise a 3′ untranslated region comprising a DNA segment that contains a polyadenylation signal and any other regulatory signals capable of effecting mRNA processing or gene expression. The vector construct described herein may also include enhancers, either translational or transcriptional enhancers, as may be required.

Also disclosed is a method of correcting movement impairments due to a neurological disease in a patient in need thereof comprising administering a therapeutically effective amount of a CRP40 polypeptide fragment as defined herein, or a polynucleotide encoding a CRP40 polypeptide fragment as defined herein. In one embodiment, the method comprises administering to the patient a therapeutically effective amount of a human CRP40 fragment comprising the amino acid sequence as defined in SEQ ID NO: 5, or a functionally equivalent variant thereof exhibiting at least about 80% sequence identity with the amino acid sequence of SEQ ID NO:5, or a nucleic acid encoding said CRP40 fragment or functionally equivalent variant.

The CRP40 polypeptide fragments and polynucleotides encoding them may be administered by any suitable route of administration and in any suitable dosage form. Methods of administration may include, but are not limited to, intradermal, intrapulmonary, intramuscular, intraperitoneal, intravenous, subcutaneous, intranasal, intrathecal, epidural, and oral routes. The compounds or compositions may be administered by any convenient route, for example by infusion or injection. Administration and may be administered together with other biologically active agents and may be systemic or local.

In some embodiments, the CRP40 polypeptide fragments and polynucleotides encoding them may be administered directly to the CNS. The pharmaceutical compounds or compositions of the invention may be administered into the central nervous system by any suitable route. In a specific example, treatment provides a compound and/or composition as described herein to the tissues of the CNS by administration directly into the cerebrospinal fluid (CSF). Means of delivery to the CSF and brain include, but are not limited to intrathecal (IT), intracerebroventricular (ICV), and intraparenchymal administration. IT or ICV administration may be achieved through the use of surgically implanted pumps that infuse the therapeutic agent into the cerebrospinal fluid.

Intraparenchymal delivery may be achieved by the surgical placement of a catheter into the brain. As used herein, “delivery to the CSF” and “administration to the CSF” encompass the IT infusion or ICV infusion of a compound and/or composition as described herein through the use of an infusion pump. In some embodiments, IT infusion is a suitable means for delivery to the CSF. In other embodiments, a compound and/or composition as described herein is continuously infused into the CSF for the entire course of treatment; such administration is referred to as “continuous infusion” or, in the case of IT infusion, “continuous IT infusion.” Also contemplated is continuous intraparenchymal infusion using a pump.

There are a variety of emerging technologies that could be used to safely and effectively administer the CRP40 fragments or polypeptides encoding them to the specific midbrain regions affected in PD or other neurological disorders. For example, it is possible to directly administer CRP40 or its functional fragments to the brain by injecting said fragments. Additional methods include drug pump delivery systems (intrathecal delivery) that employ a pump and a specifically designed catheter implanted under the skin to deliver drugs across the blood-brain barrier and increase distribution to targeted brain regions (Gill et al., 2003). As well, there are also a variety of non-invasive options for delivery of large peptides into the brain. Trans-vascular delivery of large peptides, including GDNF (˜211 amino acids) across the blood-brain barrier has been successfully achieved using the insulin receptor and the natural process of receptor-mediated transcytosis (Boado et al., 2008; Pardridge, W. M. 2008; and Pardridge and Boado, 2009). The human insulin receptor of the blood-brain barrier normally functions in receptor-mediated transcytosis of endogenous insulin (Pardridge and Boado, 2009).

Large therapeutic peptides can be re-engineered as fusion proteins with a chimeric monoclonal antibody to the human insulin receptor, which can act as a molecular Trojan horse to deliver the therapies across the blood-brain barrier without interfering with insulin transport (Pardridge and Boado, 2009). Over the past few decades, peptidic nanoparticles have emerged as superior delivery vehicles for drugs across the blood-brain barrier (Malavolta and Cabral, 2011). These nanosystems can be used to deliver small peptides (˜7 amino acids) across the blood-brain barrier (Costantino et al., 2005). There are numerous advantages of using nanosystems over other drug delivery methods across the blood-brain barrier, including: improved transport properties and drug efficacy with reduced drug toxicity; maintenance of drug stability through protection from degradation; greater control of loading and release of cargo; and the ability to incorporate molecular-targeting factors to increase distribution to targeted brain regions (Malavolta and Cabral, 2011).

The present disclosure also relates to pharmaceutical compositions comprising a CRP40 fragment as described herein, and a pharmaceutically acceptable diluent or carrier. In some embodiments, the composition may comprise a CRP40 fragment as described herein. In some embodiments, the composition may comprise nucleic acid molecule encoding a CRP40 fragment as described herein. The nucleic acid molecule may, for example, be in a vector capable of expressing the CRP40 fragment.

As used herein, “pharmaceutical composition” comprises a pharmacologically effective amount of a compound and/or composition and a pharmaceutically acceptable carrier. The pharmaceutical compositions and formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product. As used herein, “pharmaceutically acceptable diluent or carrier” refers to a diluent or carrier for administration of the compound and/or composition. Acceptable diluents and carriers are well known to the skilled worker. Selection of a diluent or carrier is based on a number of factors, including but not limited to, the solubility of the compound and the route of administration. Such considerations are well understood by the skilled worker. In one example, such carriers include, but are not limited to, saline, buffered saline, dextrose, water, glycerol, ethanol, and combinations thereof.

Antibodies against the CRP40 fragments described herein are also provided in another aspect. The antibodies may be monoclonal or polyclonal. Conventional methods can be used to prepare the antibodies. These methods are known to a person of skill in the art.

The term “antibody” as used herein is intended to include fragments thereof which also are prepared against a CRP40 fragment disclosed herein.

TABLE 1
Sequence information for SEQ ID NO's 1-52

| SEQ | | | |
|---|---|---|---|
| ID | | | |
| NO: | Name | Sequence | comment |
| 1 | CRP40 | MDSSGPKHLNMKLTRAQFEGIVTDLIRRTIAPCQKAMQDAEVSKSDIGEVI | polypeptide |
| | | LVGGMTRMPKVQQTVQDLFGRAPSKAVNPDEAVAIGAAIQGGVLAGDV | |
| | | TDVLLLDVTPLSLGIETLGGVFTKLINRNTTIPTKKSQVFSTAADGQTQVEIK | |
| | | VCQGEREMAGDNKLLGQFTLIGIPPAPRGVPQIEVTFDIDANGIVHVSAKD | |
| | | KGTGREQQIVIQSSGGLSKDDIENMVKNAEKYAEEDRRKKERVEAVNMA | |
| | | EGIIHDTETKMEEFKDQLPADECNKLKEEISKMRELLARKDSETGENIRQAA | |
| | | SSLQQASLKLFEMAYKKMASEREGSGSSGTGEQKEDQKEEKQ | |
| 2 | P1P3 | MDSSGPKHLNMKLTRAQFEGIVTDLIRRTIAPCQKAMQDAEVSKSDIGEVI | polypeptide |
| | | LVGGMTRMPKVQQTVQDLFGRAPSKaVNPDEAVAIGAAIQGGVLA | |
| 3 | P1P4 | MDSSGPKHLNMKLTRAQFEGIVTDLIRRTIAPCQKAMQDAEVSKSDIGEVI | polypeptide |
| | | LVGGMTRMPKVQQTVQDLFGRAPSKAVNPDEAVAIGAAIQGGVLAGDV | |
| | | TDVLLLDVTPLSLGIETLGGVFTKLINRNTTIPTKKSQVFSTAADGQTQVE | |
| 4 | P1P5 | MDSSGPKHLNMKLTRAQFEGIVTDLIRRTIAPCQKAMQEVSKSDIGEVILV | polypeptide |
| | | GGMTRMPKVQQTVQGLFGRAPSKAVNPDEAVAIGAAIQGGVLAGDVT | |
| | | DVLLLDVTPLSLGIETLGGVFTKLINRNTTIPTKKSQVFSTAADGQTQVEIKV | |
| | | CQGEREMAGDNKLLGQFTLIGIPPAPRGVPQIEVTFDIDANGIVHVSAKDK | |
| | | GTGRERQIVIQSSGGLSKDDIEENMVKNAEKYAEEDRRKKERV | |
| 5 | P2P4 | LAGDVTDVLLLDVTPLSLGIETLGGVFTKLINRNTTIPTKKSQVFSTAADGQT | polypeptide |
| | | QVEIKVC | |
| 6 | P2P5 | VLAGDVTDVLLLDVTPLSLGIETLGGVFTKLINRNTTIPTKKSQVFSTAADG | polypeptide |
| | | QTQVEIKVCQGEREMAGDNKLLGQFTLIGIPPAPRGVPQIEVTFDIDANGI | |
| | | VVHVSAKDKGTGRERQIVIQSSGGLSKDDIENMVKNAEKYAEEDRRKKER | |
| | | V | |
| 7 | SYN | QDAEVSKSDIGEVILVGGMTRMPKVQQTVQDLFGRAPSKAVNPDEAVA | polypeptide |
| 8 | P2P4 | TTGGCCGGCGATGTCACGGATGTGCTGCTCCTTGATGTCACTCCCCTGT | Nucleic acid |
| | | CTCTGGGTATTGAAACTCTAGGAGGTGTCTTTACCAAACTTATTAATAG | |
| | | GAATACCACTATTCCAACCAAGAAGAGCCAGGTATTCTCTACTGCCGCT | |
| | | GATGGTCAAACGCAAGTGGAAATTAAAGTGTGT | |
| 9 | β1-4 | LAGDVTDVLLLDVTPLSLGIETLGGVFTKLINRNTTIPTKKSQVFSTAA | polypeptide |
| 10 | β 1-3 | LAGDVTDVLLLDVTPLSLGIETLGGVFTKLINRNTT | polypeptide |
| 11 | β 1-2 | LAGDVTDVLLLDVTPLSLGIETLG | polypeptide |
| 12 | β 1-1 | LAGDVTDVLLLDVTP | polypeptide |
| 13 | β 2-5 | PLSLGIETLGGVFTKLINRNTTIPTKKSQVFSTAADGQTQVEIKV | polypeptide |
| 14 | β 2-4 | PLSLGIETLGGVFTKLINRNTTIPTKKSQVFSTAA | polypeptide |
| 15 | β 2-3 | PLSLGIETLGGVFTKLINRNTT | polypeptide |
| 16 | β 2-2 | PLSLGIETLG | polypeptide |
| 17 | β 3-5 | GGVFTKLINRNTTIPTKKSQVFSTAADGQTQVEIKVC | polypeptide |
| 18 | β 3-4 | GGVFTKLINRNTTIPTKKSQVFSTAA | polypeptide |
| 19 | β 3-3 | GGVFTKLINRNT | polypeptide |
| 20 | β 4-5 | NTTIPTKKSQVFSTAADGQTQVEIKV | polypeptide |
| 21 | β 4-4 | NTTIPTKKSQVFSTA | polypeptide |
| 22 | β 5-5 | GQTQVEIKVC | polypeptide |
| 23 | B1F | 5′ TAGGGATCCTTGGCCGGCGATGTCACGGATGTG 3′ | Forward primer |
| 24 | B2F | 5′ TAGGGATCCCCCCTGTCTCTGGGTATTGA 3′ | Forward primer |
| 25 | B3F | 5′ TAGGGATCCGGAGGTGTCTTTACCAAACTTATTAATAG 3′ | Forward primer |
| 26 | B4F | 5′ TAGGGATCCAATACCACTATTCCAACCAAGAAGAG 3′ | Forward primer |
| 27 | B5F | 5′ TAGGGATCCGGTCAAACGCAAGTGGAAAT 3′ | Forward primer |
| 28 | E1R | 5′ CTAGAATTCTCAGGGAGTGACATCAAGGAGCA 3′ | Reverse primer |
| 29 | E2R | 5′ CTAGAATTCTCATCCTAGAGTTTCAATACCCAGAGAC 3′ | Reverse primer |
| 30 | E3R | 5′ CTAGAATTCTCAAGTGGTATTCCTATTAATAAGTTTGGTAAA | Reverse primer |
| 31 | E4R | 5′ CTAGAATTCTCAAGCGGCAGTAGAGAATACCTG 3′ | Reverse primer |
| 32 | β 1-4 | TTGGCCGGCGATGTCACGGATGTGCTGCTCCTTGATGTCACTCCCCTGT | Nucleic acid |
| | (nuc) | CTCTGGGTATTGAAACTCTAGGAGGTGTCTTTACCAAACTTATTAATAG | |
| | | GAATACCACTATTCCAACCAAGAAGAGCCAGGTATTCTCTACTGCCGC | |
| 33 | β1-3 | TTGGCCGGCGATGTCACGGATGTGCTGCTCCTTGATGTCACTCCCCTGT | Nucleic acid |
| | (nuc) | CTCTGGGTATTGAAACTCTAGGAGGTGTCTTTACCAAACTTATTAATAG | |
| | | GAATACCACT | |
| 34 | β1-2 | TTGGCCGGCGATGTCACGGATGTGCTGCTCCTTGATGTCACTCCCCTGT | Nucleic acid |
| | (nuc) | CTCTGGGTATTGAAACTCTAGGA | |
| 35 | β1-1 | TTGGCCGGCGATGTCACGGATGTGCTGCTCCTTGATGTCACTCCC | Nucleic acid |
| | (nuc) | | |
| 36 | β2-5 | CCCCTGTCTCTGGGTATTGAAACTCTAGGAGGTGTCTTTACCAAACTTA | Nucleic acid |
| | (nuc) | TTAATAGGAATACCACTATTCCAACCAAGAAGAGCCAGGTATTCTCTAC | |
| | | TGCCGCTGATGGTCAAACGCAAGTGGAAATTAAAGTGTGT | |
| 37 | β2-4 | CCCCTGTCTCTGGGTATTGAAACTCTAGGAGGTGTCTTTACCAAACTTA | Nucleic acid |
| | (nuc) | TTAATAGGAATACCACTATTCCAACCAAGAAGAGCCAGGTATTCTCTAC | |
| | | TGCCGCT | |
| 38 | β2-3 | CCCCTGTCTCTGGGTATTGAAACTCTAGGAGGTGTCTTTACCAAACTTA | Nucleic acid |
| | (nuc) | TTAATAGGAATACCACT | |
| 39 | β2-2 | CCCCTGTCTCTGGGTATTGAAACTCTAGGA | Nucleic acid |
| | (nuc) | | |
| 40 | β 3-5 | GGAGGTGTCTTTACCAAACTTATTAATAGGAATACCACTATTCCAACCA | Nucleic acid |
| | (nuc) | AGAAGAGCCAGGTATTCTCTACTGCCGCTGATGGTCAAACGCAAGTGG | |
| | | AAATTAAAGTGTGT | |
| 41 | β 3-4 | GGAGGTGTCTTTACCAAACTTATTAATAGGAATACCACTATTCCAACCA | Nucleic acid |
| | (nuc) | AGAAGAGCCAGGTATTCTCTACTGCCGCT | |
| 42 | β 3-3 | GGAGGTGTCTTTACCAAACTTATTAATAGGAATACCACT | Nucleic acid |
| | (nuc) | | |
| 43 | β4-5 | AATACCACTATTCCAACCAAGAAGAGCCAGGTATTCTCTACTGCCGCTG | Nucleic acid |
| | (nuc) | ATGGTCAAACGCAAGTGGAAATTAAAGTGTGT | |
| 44 | β4-4 | AATACCACTATTCCAACCAAGAAGAGCCAGGTATTCTCTACTGCCGCT | Nucleic acid |
| | (nuc) | | |
| 45 | B5-5 | GGTCAAACGCAAGTGGAAATTAAAGTGTGT | Nucleic acid |
| | (nuc) | | |
| 46 | DNA K | GDVKDVLLLDVTPLSLGIETMGGVMTTLIAKNTTIPTKHSQVFSTAEDNQS | polypeptide |
| | (fragmt) | AVTIHVLQG | |
| 47 | CRP40 | ATGGATTCTTCTGGACCCAAGCATTTGAATATGAAGTTGACCCGTGCTC | Nucleic acid |
| | (nuc) | AATTTGAAGGGATTGTCACTGATCTAATCAGAAGGACTATCGCTCCATG | |
| | | CCAAAAAGCTATGCAAGATGCAGAAGTCAGCAAGAGTGACATAGGAG | |
| | | AAGTGATTCTTGTGGGTGGCATGACTAGGATGCCCAAGGTTCAGCAGA | |
| | | CTGTACAGGATCTTTTTGGCAGAGCCCCAAGTAAAGCTGTCAATCCTGA | |
| | | TGAGGCTGTGGCCATTGGAGCTGCCATTCAGGGAGGTGTGTTGGCCG | |
| | | GCGATGTCACGGATGTGCTGCTCCTTGATGTCACTCCCCTGTCTCTGGG | |
| | | TATTGAAACTCTAGGAGGTGTCTTTACCAAACTTATTAATAGGAATACC | |
| | | ACTATTCCAACCAAGAAGAGCCAGGTATTCTCTACTGCCGCTGATGGTC | |
| | | AAACGCAAGTGGAAATTAAAGTGTGTCAGGGTGAAAGAGAGATGGCT | |
| | | GGAGACAACAAACTCCTTGGACAGTTTACTTTGATTGGAATTCCACCAG | |
| | | CCCCTCGTGGAGTTCCTCAGATTGAAGTTACATTTGACATTGATGCCAA | |
| | | TGGGATAGTACATGTTTCTGCTAAAGATAAAGGCACAGGACGTGAGCA | |
| | | GCAGATTGTAATCCAGTCTTCTGGTGGATTAAGCAAAGATGATATTGA | |
| | | AAATATGGTTAAAAATGCAGAGAAATATGCTGAAGAAGACCGGCGAA | |
| | | AGAAGGAACGAGTTGAAGCAGTTAATATGGCTGAAGGAATCATTCAC | |
| | | GACACAGAAACCAAGATGGAAGAATTCAAGGACCAATTACCTGCTGAT | |
| | | GAGTGCAACAAGCTGAAAGAAGAGATTTCCAAAATGAGGGAGCTCCT | |
| | | GGCTAGAAAAGACAGCGAAACAGGAGAAAATATTAGACAGGCAGCAT | |
| | | CCTCTCTTCAGCAGGCATCACTGAAGCTGTTCGAAATGGCATACAAAAA | |
| | | GATGGCATCTGAGCGAGAAGGCTCTGGAAGTTCTGGCACTGGGGAAC | |
| | | AAAAGGAAGATCAAAAGGAGGAAAAACAGTAA | |
| 48 | DnaK | atgggtaaaataattggtatcgacctgggtactaccaactcttgtgtagcgattatggatggca | Nucleic acid |
| | (nuc) | ccactcctcgtgtgctggagaacgccgaaggcgatcgcaccacgccttctatcattgcctatac | |
| | | ccaggatggtgaaactctggttggtcagccggctaaacgtcaggcagtgacgaacccgcaaa | |
| | | acaccctgtttgcgattaaacgcctgattggccgccgcttccaggacgaagaagtacagcgtg | |
| | | atgtttccatcatgccgttcaaaattattgctgctgataacggcgacgcatgggtcgaagttaa | |
| | | aggccagaaaatggcaccgccgcagatctctgctgaagtgctgaaaaaaatgaagaaaacc | |
| | | gctgaagattacctgggtgaaccggtaactgaagctgttatcaccgtaccggcatactttaac | |
| | | gatgctcagcgtcaggcaaccaaagacgcaggccgtatcgctggtctggaagtaaaacgtat | |
| | | catcaacgaaccgaccgcagctgcgctggcttacggtctggacaaaggtactggcaaccgta | |
| | | ctatcgcggtttatgacctgggtggtggtactttcgatatttctattatcgaaatcgacgaagtt | |
| | | gacggcgaaaaaaccttcgaagttctggcaaccaacggtgatacccacctgggtggtgaag | |
| | | acttcgacagccgtctgatcaactacctggttgaagaattcaagaaagatcagggcattgacc | |
| | | tgcgcaacgatccgctggcaatgcagcgcctgaaagaagcggcagaaaaagcgaaaatcg | |
| | | aactgtcttccgctcagcagaccgacgttaacctgccgtacatcactgcagatgcgaccggtc | |
| | | cgaaacacatgaacatcaaagtgactcgtgcgaaactggaaagcctggttgaagatctggta | |
| | | aaccgttccattgagccgctgaaagttgcactgcaggacgctggcctgtccgtatctgatatcg | |
| | | acgacgttatcctcgttggtggtcagactcgtatgccaatggttcagaagaaagttgctgagtt | |
| | | ctttggtaaagagccgcgtaaagacgttaacccggacgaagctgtagcaatcggtgctgctgt | |
| | | tcagggtggtgttctgactggtgacgtgaaagacgtactgctgctggacgttaccccgctgtct | |
| | | ctgggtatcgaaaccatgggcggtgtgatgacgacgctgatcgcgaaaaacaccactatccc | |
| | | gaccaagcacagccaggtgttctctaccgctgaagacaaccagtctgcggtaaccatccatgt | |
| | | gctgcagggtgaacgtaaacgtgcggctgataacaaatctctgggtcagttcaacctggatg | |
| | | gtatcaacccggcaccgcgcggcatgccgcagatcgaagttaccttcgatatcgatgctgacg | |
| | | gtatcctgcacgtttccgcgaaagataaaaacagcggtaaagagcagaagatcaccatcaa | |
| | | ggcttcttctggtctgaacgaagatgaaatccagaaaatggtacgcgacgcagaagctaacg | |
| | | ccgaagctgaccgtaagtttgaagagctggtacagactcgcaaccagggcgaccatctgctg | |
| | | cacagcacccgtaagcaggttgaagaagcaggcgacaaactgccggctgacgacaaaactg | |
| | | ctatcgagtctgcactgactgcactggaaactgctctgaaaggtgaagacaaagccgctatc | |
| | | gaagcgaaaatgcaggagctggcacaggtttcccagaaactgatggaaatcgcccagcagc | |
| | | aacatgcccagcagcagactgccggtgctgatgcttctgcaaacaacgcgaaagatgacgat | |
| | | gttgtcgacgctgaatttgaagaagtcaaagacaaaaaataa | |
| 49 | DnaK | MGKIIGIDLGTTNSCVAIMDGTTPRVLENAEGDRTTPSIIAYTQDGETLVG | polypeptide |
| | (prot) | QPAKRQAVTNPQNTLFAIKRLIGRRFQDEEVQRDVSIMPFKIIAADNGDA | |
| | | WVEVKGQKMAPPQISAEVLKKMKKTAEDYLGEPVTEAVITVPAYFNDAQ | |
| | | RQATKDAGRIAGLEVKRIINEPTAAALAYGLDKGTGNRTIAVYDLGGGTFD | |
| | | ISIIEIDEVDGEKTFEVLATNGDTHLGGEDFDSRLINYLVEEFKKDQGIDLRN | |
| | | DPLAMQRLKEAAEKAKIELSSAQQTDVNLPYITADATGPKHMNIKVTRAK | |
| | | LESLVEDLVNRSIEPLKVALQDAGLSVSDIDDVILVGGQTRMPMVQKKVAE | |
| | | FFGKEPRKDVNPDEAVAIGAAVQGGVLTGDVKDVLLLDVTPLSLGIETMG | |
| | | GVMTTLIAKNTTIPTKHSQVFSTAEDNQSAVTIHVLQGERKRAADNKSLG | |
| | | QFNLDGINPAPRGMPQIEVTFDIDADGILHVSAKDKNSGKEQKITIKASSG | |
| | | LNEDEIQKMVRDAEANAEADRKFEELVQTRNQGDHLLHSTRKQVEEAGD | |
| | | KLPADDKTAIESALTALETALKGEDKAAIEAKMQELAQVSQKLMEIAQQQ | |
| | | HAQQQTAGADASANNAKDDDVVDAEFEEVKDKK | |
| 50 | P1P3 | ATGGATTCTTCTGGACCCAAGCATTTGAATATGAAGTTGACCCGTGCTC | Nucleic acid |
| | (nuc) | AATTTGAAGGGATTGTCACTGATCTAATCAGAAGGACTATCGCTCCATG | |
| | | CCAAAAAGCTATGCAAGATGCAGAAGTCAGCAAGAGTGACATAGGAG | |
| | | AAGTGATTCTTGTGGGTGGCATGACTAGGATGCCCAAGGTTCAGCAGA | |
| | | CTGTACAGGATCTTTTTGGCAGAGCCCCAAGTAAAGCTGTCAATCCTGA | |
| | | TGAGGCTGTGGCCATTGGAGCTGCCATTCAGGGAGGTGTGTTGGCC | |
| 51 | P1P4 | ATGGATTCTTCTGGACCCAAGCATTTGAATATGAAGTTGACCCGTGCTC | Nucleic acid |
| | (nuc) | AATTTGAAGGGATTGTCACTGATCTAATCAGAAGGACTATCGCTCCATG | |
| | | CCAAAAAGCTATGCAAGATGCAGAAGTCAGCAAGAGTGACATAGGAG | |
| | | AAGTGATTCTTGTGGGTGGCATGACTAGGATGCCCAAGGTTCAGCAGA | |
| | | CTGTACAGGATCTTTTTGGCAGAGCCCCAAGTAAAGCTGTCAATCCTGA | |
| | | TGAGGCTGTGGCCATTGGAGCTGCCATTCAGGGAGGTGTGTTGGCCG | |
| | | GCGATGTCACGGATGTGCTGCTCCTTGATGTCACTCCCCTGTCTCTGGG | |
| | | TATTGAAACTCTAGGAGGTGTCTTTACCAAACTTATTAATAGGAATACC | |
| | | ACTATTCCAACCAAGAAGAGCCAGGTATTCTCTACTGCCGCTGATGGTC | |
| | | AAACGCAAGTGGAA | |
| 52 | P1P5 | ATGGATTCTTCTGGACCCAAGCATTTGAATATGAAGTTGACCCGTGCTC | Nucleic acid |
| | (nuc) | AATTTGAAGGGATTGTCACTGATCTAATCAGAAGGACTATCGCTCCATG | |
| | | CCAAAAAGCTATGCAAGATGCAGAAGTCAGCAAGAGTGACATAGGAG | |
| | | AAGTGATTCTTGTGGGTGGCATGACTAGGATGCCCAAGGTTCAGCAGA | |
| | | CTGTACAGGATCTTTTTGGCAGAGCCCCAAGTAAAGCTGTCAATCCTGA | |
| | | TGAGGCTGTGGCCATTGGAGCTGCCATTCAGGGAGGTGTGTTGGCCG | |
| | | GCGATGTCACGGATGTGCTGCTCCTTGATGTCACTCCCCTGTCTCTGGG | |
| | | TATTGAAACTCTAGGAGGTGTCTTTACCAAACTTATTAATAGGAATACC | |
| | | ACTATTCCAACCAAGAAGAGCCAGGTATTCTCTACTGCCGCTGATGGTC | |
| | | AAACGCAAGTGGAAATTAAAGTGTGTCAGGGTGAAAGAGAGATGGCT | |
| | | GGAGACAACAAACTCCTTGGACAGTTTACTTTGATTGGAATTCCACCAG | |
| | | CCCCTCGTGGAGTTCCTCAGATTGAAGTTACATTTGACATTGATGCCAA | |
| | | TGGGATAGTACATGTTTCTGCTAAAGATAAAGGCACAGGACGTGAGCA | |
| | | GCAGATTGTAATCCAGTCTTCTGGTGGATTAAGCAAAGATGATATTGA | |
| | | AAATATGGTTAAAAATGCAGAGAAATATGCTGAAGAAGACCGGCGAA | |
| | | AGAAGGAACGAGTT | |
| 53 | P2P5 | GTGTTGGCCGGCGATGTCACGGATGTGCTGCTCCTTGATGTCACTCCCC | Nucleic acid |
| | (nuc) | TGTCTCTGGGTATTGAAACTCTAGGAGGTGTCTTTACCAAACTTATTAA | |
| | | TAGGAATACCACTATTCCAACCAAGAAGAGCCAGGTATTCTCTACTGCC | |
| | | GCTGATGGTCAAACGCAAGTGGAAATTAAAGTGTGTCAGGGTGAAAG | |
| | | AGAGATGGCTGGAGACAACAAACTCCTTGGACAGTTTACTTTGATTGG | |
| | | AATTCCACCAGCCCCTCGTGGAGTTCCTCAGATTGAAGTTACATTTGAC | |
| | | ATTGATGCCAATGGGATAGTACATGTTTCTGCTAAAGATAAAGGCACA | |
| | | GGACGTGAGCAGCAGATTGTAATCCAGTCTTCTGGTGGATTAAGCAAA | |
| | | GATGATATTGAAAATATGGTTAAAAATGCAGAGAAATATGCTGAAGAA | |
| | | GACCGGCGAAAGAAGGAACGAGTT | |

### EXAMPLES

The following examples are provided to assist in understanding the invention. The invention is not limited to the exemplary embodiments disclosed.

### Example 1

### Preparation of CRP40 Fragments

Preparation/Cloning Methods for Proteins

Preparation of CRP40 and controls: Recombinant CRP40 protein was synthesized as described in Gabriele et al. (2009). The heat shock protein 47 (Hsp47) clone was a gift from Dr. Ananthanarayanan (Department of Biochemistry, McMaster University) and was synthesized as described by Thomson and Ananthanarayanan (2001). Hsp47, a collagen-specific chaperone, was chosen as a negative control due to its similar size to CRP40 (e.g., the size of Hsp47 is 47 kDa, while the size of CRP40 is 40 kDa) and its role as a molecular chaperone (Thomson and Ananthananrayanan, 2001) and has now been validated as a negative control. A series of competitive radio-labelled ligand binding studies using tritiated dopamine ([3H]-DA) were performed to validate Hsp47 as a negative control. The results indicated that Hsp47 did not bind DA, unlike CRP40, which was used as a positive control because of its previously determined capability of binding DA. In addition, sequence alignment of Hsp47 and CPR40 did not identify significant regions of similarity. Artificial cerebrospinal fluid (CSF) control was prepared according to previously established protocols that are known to a person of skill in the art.

Methods for Cloning/Preparation of CRP40 Fragments:

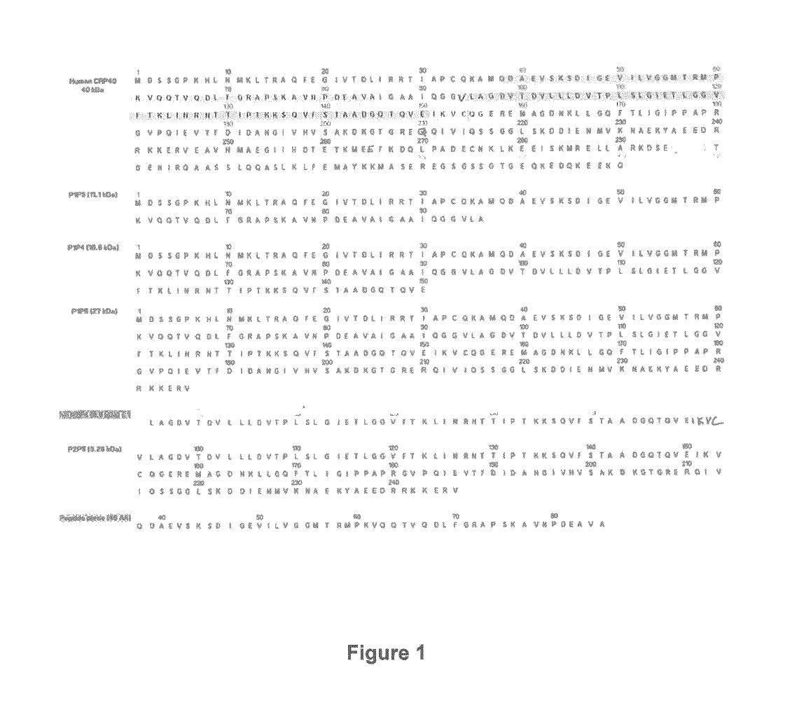

FIG. 1 shows the sequences of five fragments within the 40 kDa CRP40 protein that were identified using a bioinformatics approach, and the alignment of these fragments relative to full length CRP40. The fragments were named according to their forward and reverse primer pairs: P1P3 (11.1 kDa); P1P4 (16.6 kDa); P1P5 (27 kDa); P2P4 (6.6 kDa); and P2P5 (9.26 kDa). A peptide piece (48 amino acids) was manufactured and is also displayed.

Primers for cloning certain disclosed CRP40 fragments are outlined in Table 1, and were synthesized by MOBIX Laboratory (McMaster University). Cloning was performed with cDNA made using SHSY-5Y cells, and DNA for regions of interest was amplified using standard polymerase chain reaction (PCR) protocols optimized for CPR40 in the inventor's laboratory. The pGEX-2T vector (GE Healthcare, USA) and the protein fragments were digested using FastDigest EcoRI and FastDigest BamHI (Fermentas Inc., Canada).

TABLE 2
Forward and reverse primer sequences for
cloning CRP40 fragments
Forward primers

| | |
|---|---|
| B1F | 5′ TAG GGA TCC TTG GCC GGC GAT GTC |
| | ACG GAT GTG 3′ |
| B2F | 5′ TAG GGA TCC CCC CTG TCT CTG GGT |
| | ATT GA 3′ |
| B3F | 5′ TAG GGA TCC GGA GGT GTC TTT ACC |
| | AAA CTT ATT AAT AG 3′ |
| B4F | 5′ TAG GGA TCC AAT ACC ACT ATT CCA |
| | ACC AAG AAG AG 3′ |
| B5F | 5′ TAG GGA TCC GGT CAA ACG CAA GTG |
| | GAA AT 3′ |

Reverse primers

| | |
|---|---|
| E1R | 5′ CTA GAA TTC TCA GGG AGT GAC ATC |
| | AAG GAG CA 3′ |
| E2R | 5′ CTA GAA TTC TCA TCC TAG AGT TTC |
| | AAT ACC CAG AGA C 3′ |
| E3R | 5′ CTA GAA TTC TCA AGT GGT ATT CCT |
| | ATT AAT AAG TTT GGT AAA 3′ |
| E4R | 5′ CTA GAA TTC TCA AGC GGC AGT AGA |
| | GAA TAC CTG 3′ |

Underline = Bam H1; Bold = Eco R1; Bold/Underline = Stop

The ligated pGEX-2T vector and CRP40 protein fragments were transformed into One Shot TOP10 chemically competent BL-21 E. coli cells (Invitrogen, Canada) and grown at 37° C. Bacterial cultures were induced with isopropyl β-D-1-thiogalactopyranoside (IPTG) overnight at 14° C. Purification of CRP40 protein fragments was carried out using affinity chromatography, where Glutathione S-transferase (GST)-bound proteins were liberated from GST beads by thrombin protease cleavage. Excess salts were subsequently removed by dialyzing protein fragments against 1× Phosphate Buffered Saline (PBS) for 36 hours. DNA sequencing and sodium dodecyl sulphate polyacrylamide gel electrophoresis (SDS-PAGE) were used to confirm the identity of CRP40 fragments.

### Example 2

6-Hydroxydopamine (or 6-OHDA) animal model of dyskinesia and oxidative stress: The 6-OHDA model was chosen for this study as this model is characterized by oxidative stress and degeneration of DA neurons, similar to the condition in human idiopathic PD patients and is a well-known preclinical animal model of PD. Rats treated unilaterally with 6-OHDA exhibit the characteristic locomotor disturbances of PD including a shuffling gait, short steps and low walking velocity.

6-OHDA animals, male Sprague-Dawley rats from Charles River (Raleigh, N.C., USA), were prepared by hemi-lateral lesioning of the A9 dopaminergic pathway by injecting 6-OHDA toxin in the substantia nigra par compacta (SNc) region of the rat brain. This unilateral nigrostriatal degeneration cause assymetry in motor behaviours due to imbalanced dopimanergic activity between striata. The 6-OHDA hemi-lesioned rats displayed a characteristic full-body rotational behaviours in response to dompaminergic agonists, including apomorphine, unlike non-lesioned controls which did not rotate following apomorphine challenge. Baseline rotational behavior and locomoter testing were performed before surgical intervention.

The protocol for lesioning is synonymous with our 6-OHDA stereotaxic injection protocol found in Modi et al., (1996). Rats were sent to the McMaster University animal facility and allowed to acclimatize to the colony room for 1 week following arrival. Each rat was handled by the same researcher daily for 7 days before the start of the experiment. Animals were then tested for baseline apomorphine-induced rotations (tested during light hours), and locomotor activity (tested during dark hours) under apomorphine challenge treatment. Animals were then split into groups and injected with CRP40, CRP40 fragments and peptides, and controls. Animals were housed and tested in compliance with the guidelines described in the Guide to the Care and Use of Experimental Animals (Canadian Council on Animal Care, 1984; 1993).

Stereotaxic Surgery Protocol:

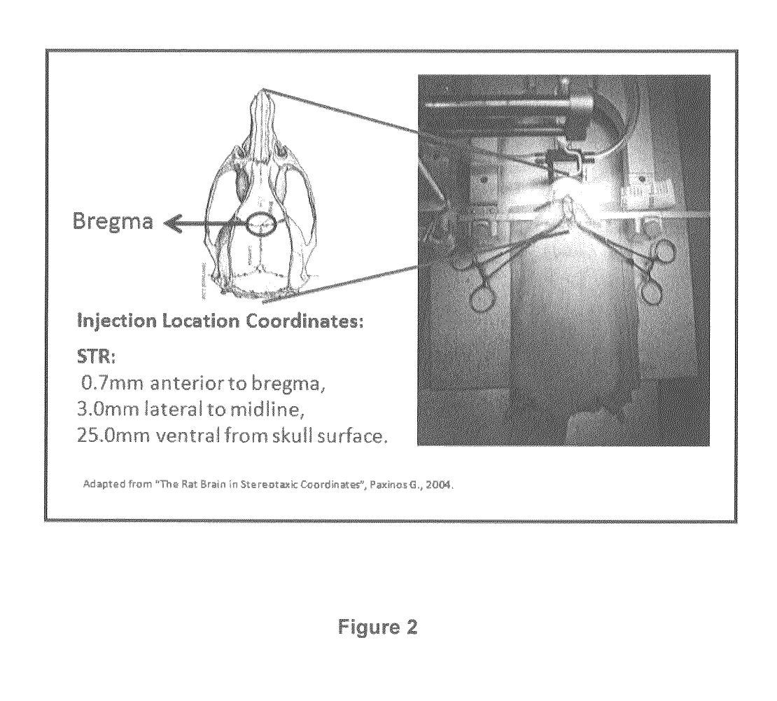

6-OHDA rats were injected in the left striatum with one of the various fragments via stereotaxic surgery. FIG. 2 shows the stereotaxic procedure for 6-OHDA rats. The left side of the diagram shows a schematic of a Sprague-Dawley rat skull with location of Bregma. Coordinates for striatal injections of treatment are as follows: A/P +0.7 mm, M/L +3.0 mm, and D/V −5.0 mm. A 25 μl Hamilton syringe with a 22 gauge 2 inch blunt needle was used to inject treatments at a rate of 1 μl/min. The right hand side of FIG. 2 shows a photo of a male Sprague-Dawley rat situated in the stereotax for surgery.

Testing rotational behaviour in 6-OHDA rats: As previously discussed, 6-OHDA has been found to deplete the neurotransmitter content of dopaminergic neurons. Apomorphine injection causes full body rotational behaviour in the direction contralateral to the 6-OHDA lesion. The total number of rotations per fixed time period is used as a measure of improving or worsening symptomology. A holding chamber in which the animal is confined to a circular shaped floor is used to count rotations, as this is an optimal arrangement for these experiments compared to an open field box. 0.5 mg/ml Apomorphine (Sigma: A4393) was dissolved in 0.9% saline with 0.1% ascorbic acid. Animals were injected subcutaneously with apomorphine (0.5 mg/kg body weight). Animals were placed in a clear plastic cylindrical holding tank and allowed 5 minutes for habituation. Following habituation, 10 minutes was allotted and a cell counter was used to record the number of rotations.

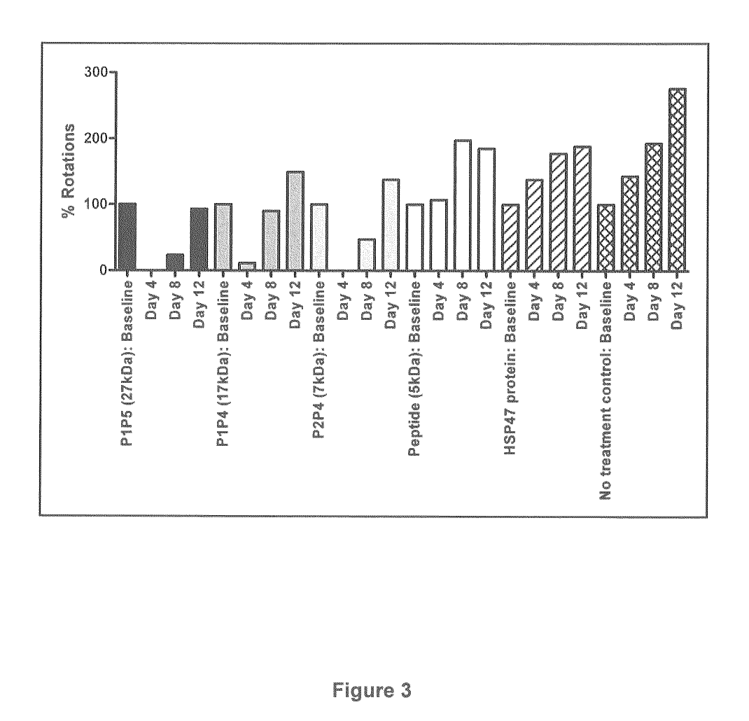

FIG. 3 shows the results of injection of P1P5, P1P4, P2P4, and the peptide piece in the 6-OHDA rat model. A reduction of rotational behavior was seen in animals treated with the P1P5, P1P4, and P2P4 fragments of CRP40 than at baseline, when compared to animals treated with controls (Hsp47; no treatment). In marked contrast, the peptide piece (48AA), that did not fall within the P2P4 region did not correct rotational behaviour. The fragment P2P5 was not tested but is also predicted to have activity since it contains the P2P4 fragment, which was found to have activity.

### Example 2

### Assessment of the Effect of CRP40 on the Behavioural Symptoms in a Rat Model of CNS Demyelination Called Long-Evans Shaker (LES)

Due to the complete demyelination of the brain and spinal cord, LES animals show tremors beginning at 10 to 12 days of age (Delaney et al., 1995). Other symptoms become apparent by 5 to 14 weeks: ataxia, hind limb paresis, and seizures (Delaney et al., 1995). Neuronal degeneration and an absence of myelinated axons can been seen in the cortex, hippocampus, and midbrain of LES rats. Despite this severe myelin deficiency, some mutants live beyond one year of age (Kwiecien et al, 1998). In situ hybridization studies demonstrate that the numbers of mature oligodendrocytes are similar to controls early in life and increase with time, However, the LES oligodendrocytes are permanently disabled and fail to produce myelin beyond the first weeks of life (Kwiecien et al., 1998). The Long Evans shaker rat represents a novel myelin mutant with an autosomal recessive mode of inheritance (Delaney et al., 1995).

Using this LES model, it was tested whether CRP40 functions in conjunction with glial cells, and whether its therapeutic effects on the function of degenerated areas in the 6-OHDA hemi-PD rat model are in some way linked to glial cell function and myelin.

This study included LES rats (n=4) and Long-Evans control rats (n=4). Because of their fragile nature, the LES rats cannot be handled regularly. In order to keep confounds to a minimum, the Long-Evans controls were not handled by experimenters either. All animals were first subjected to a series of behavioural tests for baseline measures. Tests included prepulse inhibition (PPI) and locomotor activity. The animals were also placed in cylindrical Plexiglas containers and videotaped for visual documentation of their behaviour before intervention.

Surgical Injection

Once all baseline data was collected, the rats were treated with gaseous anaesthesia, placed in a standard rat stereotax, and injected into either the lateral ventricle (from bregma: A/P −0.5 mm, M/L −1.5 mm, D/V −4.7 mm) (LES n=2 females, control n=2 females) or the striatum (from bregma: A/P +0.7 mm, M/L +3.0 mm, D/V −5.0 mm) (LES n=2 males, control n=2 males) with 100 ug CRP40 plasmid DNA. Injections were completed using a 25 uL Hamilton syringe at 1 ul/minute.

After recovery from surgery, all animals were again tested using the same behavioural methods for post-intervention measures. PPI and locomotor activity were tested at days 4, 8, and 11 post-injection. Animals were also videotaped at each time point. On day 12 post-injection, all rats were sacrificed. 1 striatal injected LES male, 1 lateral ventricle injected LES female, 1 striatal injected control male, and 1 lateral ventricle injected control female were sacrificed by perfusion and brain tissue was excised along with the spinal cord, fixed, and then prepared onto slides and analyzed for fluorescence by confocal microscope. 1 striatal injected LES male, 1 lateral ventricle injected LES female, 1 striatal injected control male, and 1 lateral ventricle injected control female were sacrificed by isoflurane overdose and decapitation by guillotine. Brain tissue was sliced using razor blades and a brain mould, then micro-punched for specific brain area samples, and flash frozen on dry ice for later analysis.

The video documentation of the peptide or CRP 40 fragments in the lateral ventricle injected female LES rats showed a definite trend for improved balance, coordination, and willingness to travel and explore, whereas the control Shaker rats are relatively inactive and do not have much functional control. After treatment with CRP40 fragments and peptides the Shaker rats see, to have some control of their movements, and even groom themselves, which are significant behavioral changes compared to the wild type rats.

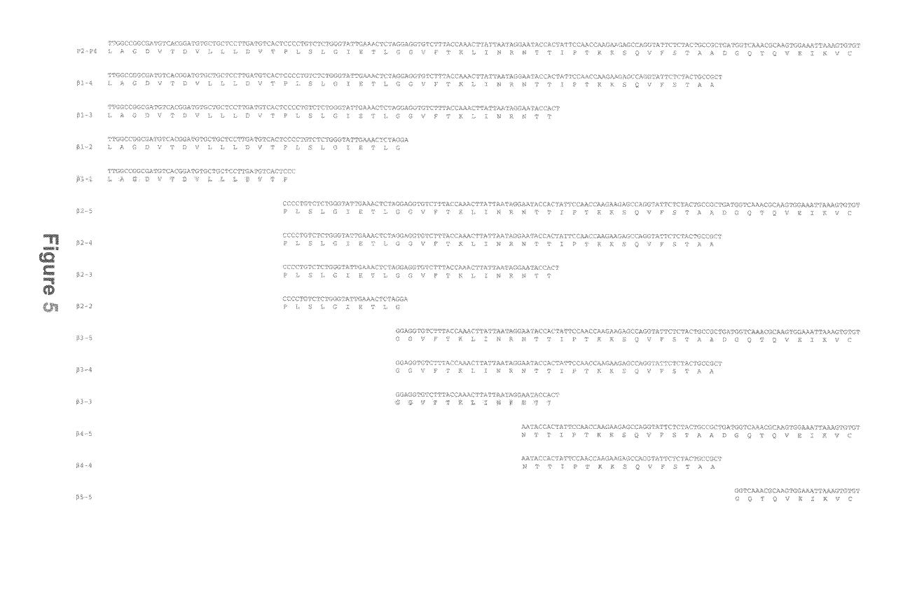

FIG. 4 shows that alignment of the protein sequence of the P2P4 fragment of CRP40 with the sequence of DnaK, a member of the 70 kDa heat shock protein family, showed an 83.3% identity. Secondary structure prediction of P2P4 (shown above the sequence) displays the location of 5 β strands, which show a high level of similarity in size and location to the 6 β-strands obtained from the crystal structure of DnaK (displayed below the sequences). Above the secondary structure prediction is additional information showing the predicted phosphorylation sites on P2P4 by protein kinase A, protein kinase C, and casein kinase 1. Protein fragments of P2P4 shown in the bottom portion of the figure (e.g., β 1-4, β 1-3, β 1-2, β 1-1, β 2-5, β 2-4, β 2-3, β 2-2, β 3-5, β 3-4, β 3-3, β 4-5, β 4-4 and β 5-5). The fragments designated based on these structural and bioinformatic analyses were cloned, expressed and purified as described above. The sequences of the fragments are outlined in FIG. 5 and in Table 1. FIG. 5 shows the alignment of the nucleic acid and amino acid sequences of the P2P4 fragments described above.

### Example 5

### Binding Assays with Catecholamine-Regulated Protein 40 (CRP40) and the 7 kDa P2P4 Fragment of CPR40

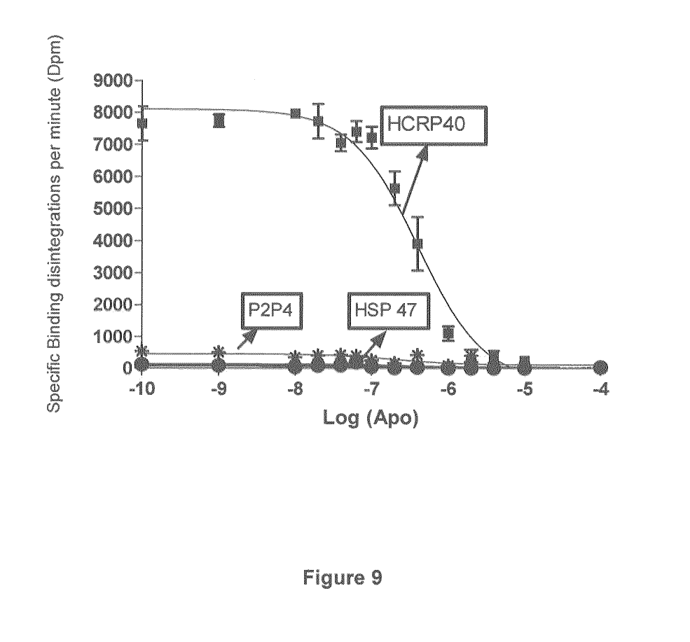

This study was conducted as a follow-up to earlier findings that showed that CRP40 and its smaller fragment, the 7 kDa P2P4, alleviate rotational symptoms in the 6-hydroxydopamine (6-OHDA) rats, a model of dyskinesia which is often used as a model of PD. The goal of this study was to determine whether the correction of rotational behaviour was due to the binding of dopamine (DA), a catecholamine implicated in neurological disorders. The study was conducted using competitive radioligand binding assays with tritiated dopamine in the presence of cold dopamine and apomorphine. Unlike CRP40, which binds dopamine, the P2P4 fragment of CRP40, which was found to correct behavioural impairments in 6-OHDA rats, does not bind dopamine, suggesting an alternative mechanism of action for its therapeutic efficacy (FIG. 8; FIG. 9.). The methodology and results are summarized below.

Methods. The ability of cold DA to displace bound tritiated dopamine ([3H]-DA) in CRP40, P2P4, or heat-shock protein 47 (a control heat-shock protein that plays a role in collagen synthesis) was assessed by competitively binding [3H]-DA in the presence of different concentrations of unlabeled DA or apomorphine (in 0.1% ascorbic acid) using the same methodology as in (Gabriele et al., 2009). Briefly, the binding of [3H]-DA was carried out in triplicate in 1.0 ml of assay buffer containing 1 nM of radioligand, across a range of concentrations of unlabeled DA and 10 μg of one of CRP40 protein, P2P4 fragment, or HSP47. For amomorphine, the binding of [3H]-DA was carried out in triplicate in 1.0 ml of assay buffer containing 1 nM of radioligand, across a range of concentrations of unlabeled apomorphine and 10 μg of one of CRP40 protein, P2P4 fragment, or HSP47. At the end of incubation, the bound and free ligands were separated by vacuum filtration through Whatman GF/B filters. The filters were washed with 3×5 ml of Tris-EDTA buffer and the radioactive counts were determined on a Beckman scintillation counter. The Assay Buffer was prepared by adding the following components to 500 ml of double distilled water: 25 ml 1M Tris-HCl (pH 8; 50 mM), 0.5 ml 500 mM EDTA (1 mM), 100 mM PMSF in ethanol (0.1 mM), 0.508 g MgCl2·6H2O (5 mM), 0.0077 g DTT (0.1 mM), 0.050 g Bacitracin (100 μg/ml), and 0.0025 g

Soybean Trypsin (5 μg/ml). The final pH was adjusted to 7.4. The filtration Buffer was prepared by adding the following components to 2 L of double distilled water: 100 ml 1M Tris-HCl (pH 8; 50 mM), and 4 ml 500 mM EDTA (1 mM). The final pH was adjusted to 7.4. Statistical analysis of the data was performed utilizing Graph Pad Prism 6.0 software. A one-way analysis of variance (ANOVA) with a confidence interval of 95% was utilized to analyze the data; the post-test used was Tukey's test.

Results. It was confirmed that CRP40 binds DA with high capacity and low affinity, as previously reported (FIG. 8; Gabriele et al., 2009), and also binds apomorphine (FIG. 9.). The P2P4 fragment does not bind either of DA (FIG. 8.) or apomorphine (FIG. 9.). The HSP47 protein (negative control) does not bind either of DA (FIG. 8.) or apomorphine (FIG. 9.), and does not elicit a correction of rotational behaviour in 6-OHDA rats.

Conclusions. The correction of rotational behaviour in 6-OHDA rats is not due to catecholamine (e.g., dopamine) binding. The P2P4 fragment may be the functional region of CRP40 that corrects rotational behaviour based on an alternative mechanism. Without wishing to be bound by theory, this mechanism may involve protection from oxidative stress in the mitochondria.

### Example 3

### Cells Overexpressing CRP40 and Mortalin have Differential Effects on Mitochondrial ROS Levels

The ability of CRP40- and mortalin-transfected SH-SY5Y cells to protect against the harmful effects of mitochondrial ROS in comparison to normal non-transfected SH-SY5Y cells was measured using the OxiSelect™ Intracellular ROS Assay Kit (Green Fluorescence). H₂O₂ treatment was used to induce oxidative stress in the assay. Increased fluorescence in this assay corresponded to greater ROS levels.

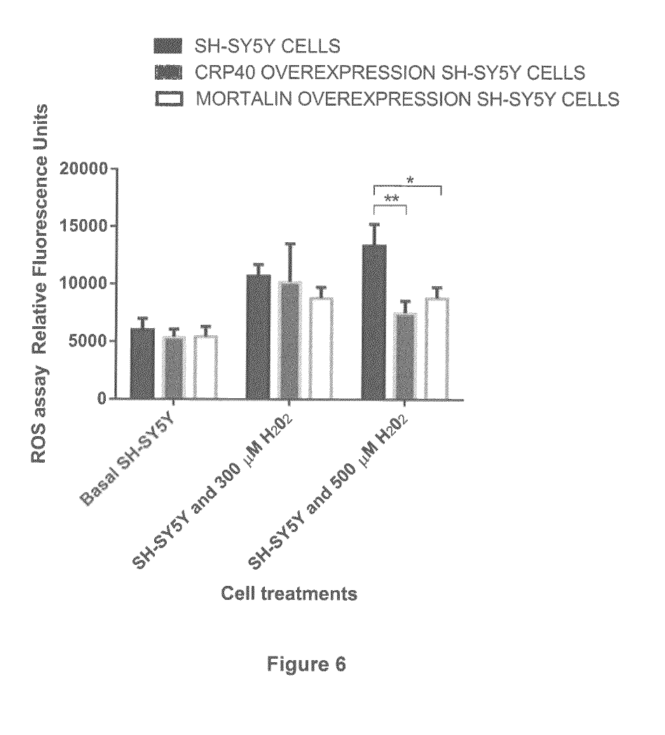

Overall, treatment with oxidant resulted in higher ROS production in normal SH-SY5Y cells, CRP40-transfected SH-SY5Y cells, and mortalin-transfected SH-SY5Y cells in comparison to the basal untreated condition (FIG. 6). Specifically, in the basal condition, where non-transfected SH-SY5Y cells were not treated with an oxidant, the levels of ROS were significantly lower than in the treated conditions for non-transfected SH-SY5Y cells (p=0.0343 for 300 μM H₂O₂ treatment and p<0.0001 for 500 μM H₂O₂ treatment). Also, CRP40-transfected SH-SY5Y cells treated with 300 μM H₂O₂ treatment had significantly higher levels of ROS than untreated CRP40-transfected SH-SY5Y cells (p=0.0343). Mortalin-transfected SH-SY5Y cells in the basal condition displayed a similar tendency for decreased ROS levels in comparison to mortalin-transfected SH-SY5Y cells treated with either 300 μM H₂O₂ or 500 μM H₂O₂.

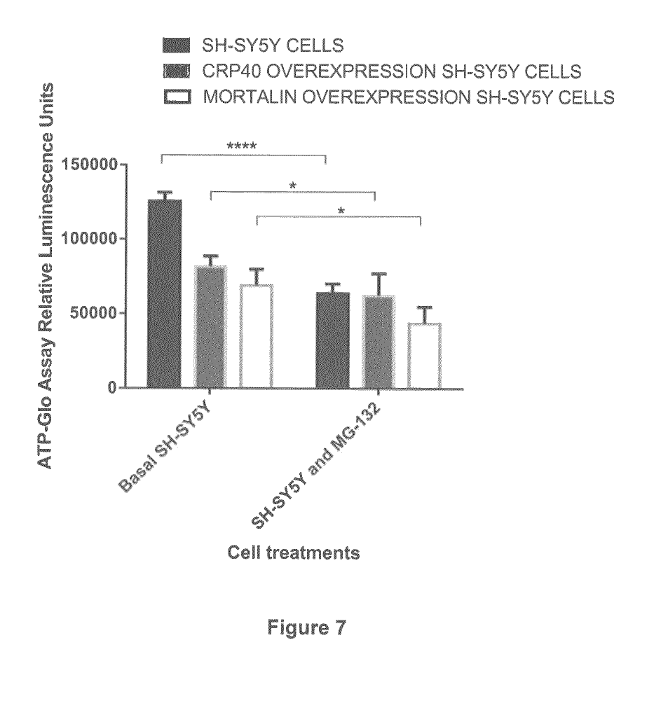

Furthermore, in the untreated condition, the normal SH-SY5Y cells showed a tendency towards slightly higher ROS levels than both the CRP40-transfected SH-SY5Y cells, and the mortalin-transfected SH-SY5Y cells (FIG. 7). Similarly, when different cells (normal SH-SY5Y cells; CRP40-transfected SH-SY5Y cells; and mortalin-transfected SH-SY5Y cells) were treated with 300 μM H₂O₂, there was a tendency towards higher ROS levels in the normal SH-SY5Y cells compared to the other conditions. The protective role of CRP40 and mortalin was observed in the 500 μM H₂O₂ treatment condition, where there was a statistically significant difference between normal SH-SY5Y cells treated with 500 μM H₂O₂ versus CRP40-transfected SH-SY5Y cells treated with 500 μM H2O2 (p=0.0056), and normal SH-SY5Y cells treated with 500 μM H₂O₂ versus mortalin-transfected SH-SY5Y cells treated with 500 μM H₂O₂ (p=0.0343). Therefore, normal SH-SY5Y cells had increased levels of ROS in comparison to CRP40- and mortalin-transfected SH-SY5Y cells, that was more pronounced when a high amount of oxidant was used to induce oxidative stress. Also, in the 500 μM H₂O₂ treatment condition, there was no statistically significant difference in ROS levels between the CRP40-transfected SH-SY5Y cells and mortalin-transfected SH-SY5Y cells, although the CRP40-transfected SH-SY5Y cells displayed a tendency towards lower ROS production in this condition.

Interestingly, when a greater concentration of H₂O₂ (e.g., 500 μM) was applied to CRP40-transfected SH-SY5Y cells, these cells showed a tendency towards higher ROS production than the untreated CRP40-transfected SH-SY5Y cells, which was the expected result. However, ROS production in the CRP40-transfected SH-SY5Y cells treated with 500 μM H₂O₂ was lower than for the CRP40-transfected SH-SY5Y cells treated with 300 μM H₂O₂. While there was a statistically significant increase in ROS levels of the untreated CRP40-transfected SH-SY5Y cells in comparison to the CRP40-transfected SH-SY5Y cells treated with 300 μM H₂O₂ (p=0.0343), there was no statistically significant difference between ROS levels in untreated CRP40-transfected SH-SY5Y cells in comparison to the CRP40-transfected SH-SY5Y cells treated with a higher concentration of H2O2 (e.g., 500 μM). This result suggests that CRP40 may have conferred a differential degree of protection against higher concentrations of the H₂O₂ oxidant, keeping ROS levels closer to those observed in the normal, untreated condition for cells transfected with CRP40.

### Example 4

### Cells Overexpressing CRP40 and Mortalin have Differential Effects on Intracellular ATP Levels

The ability of CRP40- and mortalin-transfected SH-SY5Y cells to maintain mitochondrial homeostasis, specifically energy production and ATP levels, was examined using the ATP-Glo™ Bioluminometric Cell Viability Assay Kit. MG-132 was used to inhibit proteasomal function and cause increased oxidative stress, which would compromise mitochondrial function, as well as the processes of energy production. The quantitation of ATP levels was thus used as a measure of cell viability.

FIG. 7 summarizes the results of the adenosine triphosphate assays. The left panel shows ATP levels under basal conditions in normal SH-SY5Y cells, cells overexpressing CRP40, and cells overexpressing mortalin. Mortalin was used as a positive control in this assay. The right panel shows ATP levels under conditions of proteasomal stress induced by treatment with MG-132. After treatment with proteasomal inhibitor, cells overexpressing CRP40 and mortalin demonstrated slight, but statistically significant alterations in ATP/cell viability (*p<0.05; ˜24% reduction of cell viability in CRP40-transfected cells; ˜37% reduction of cell viability in mortalin-transfected cells). In contrast, non-transfected SH-SY5Y cells demonstrated a significant drop in ATP and cell viability after treatment with MG-132 (****p<0.0001; ˜50% reduction in cell viability). The data are representative of mean±S.D. of experiments for each condition performed twice, in triplicate.

As seen in the basal condition in FIG. 7, cell viability was significantly lower in SH-SY5Y cells that have been transfected with either CRP40 or mortalin, and that overexpress these proteins, versus normal non-transfected SH-SY5Y cells (p<0.0001 for both CRP40- and mortalin-transfected cells). It is expected that ATP levels in transfected cells would not be reduced to this extent, especially in the untreated condition. The cells overexpressing either CRP40 or Mortalin should have the same viability as normal SH-SY5Y cells. The cell viability of non-transfected SH-SY5Y cells treated with MG-132 diminished significantly in comparison to the non-transfected, non-treated SH-SY5Y cells (p<0.0001). In terms of percentage, there was approximately a 50% drop in cell viability when non-transfected SH-SY5Y cells are treated with MG-132. When comparing the effect of MG-132 treatment on the viability of CRP40-transfected SH-SY5Y cells, there was a slight (˜24%), but statistically significant drop in ATP following treatment with the proteasomal inhibitor (FIG. 4.3). For mortalin-transfected SH-SY5Y cells, there was a statistically significant reduction in cell viability (˜37%) following treatment with MG-132 (FIG. 4.3). Thus, MG-132 treatment caused a reduction in cell viability of approximately 50% for normal non-transfected cells, approximately 24% for SH-SY5Y cells overexpression CRP40, and approximately 37% for cells overexpressing mortalin.

The reduction in ATP and the resulting loss of cell viability in CRP40 and mortalin-transfected cells that have been treated with MG-132 was thus not as pronounced as the loss in cell viability in the normal non-transfected cells treated with MG-132. These findings show that overexpression of CRP40 and mortalin in SH-SY5Y cells helps to maintain intracellular ATP levels and energy production from the mitochondria, and that these proteins may confer protection from cell death induced by oxidative stress.

In the preceding description, for purposes of explanation, numerous details are set forth in order to provide a thorough understanding of the embodiments. However, it will be apparent to one skilled in the art that these specific details are not required.

The above-described embodiments are intended to be examples only. Alterations, modifications and variations can be effected to the particular embodiments by those of skill in the art without departing from the scope, which is defined solely by the claims appended hereto.

References referred to throughout the description are incorporated herein by reference.

### REFERENCES

A listing of references mentioned in the description is provided below:
1. Beaulieu J M, and Gainetdinov R R. (2011). The Physiology, Signaling, and Pharmacology of Dopamine Receptors. Pharmacol Rev. 63:1182-1121.
2. Boado, R. J. et al. GDNF fusion protein for targeted-drug delivery across the human blood-brain barrier. Biotechnol. Bioeng. 100, 387-396 (2008).
3. Chang H C, Tang Y C, Hayer-Hartl M, and Hartl F U. (2007). SnapShot: molecular chaperones, Part I. Cell. 128-212.
4. Choi J, Forster M J, McDonald S R, Weintraub S T, Carroll C A, and Gracy R W. (2004). Proteomic identification of specific oxidized proteins in ApoE-knockout mice: relevance to Alzheimer's disease. Free Radic Biol Med. 36:1155-1162.
5. Costantino, L. et al. Peptide-derivatized biodegradable nanoparticles able to cross the blood-brain barrier. J. Control Release. 108(1), 84-86 (2005).
6. Delaney K H, Kwiecien J M, Wegiel J, Wisniewski H M, Percy D H, Fletch A L. Familial dysmyelination in a Long Evans rat mutant. Lab Anim Sci. Oct; 45(5):547-53 (1995).
7. Deocaris C C, Kaul S C, and Wadhwa R. (2008). From proliferative to neurological role of an hsp70 stress chaperone, mortalin. Biogerontol. 9(6):391-403.
8. Ellis, J. (1987). Proteins as molecular chaperones. Nature. 328:378-379.
9. Farrer M J. (2006). Genetics of Parkinson disease: paradigm shifts and future prospects. Nat Rev Genet. 7:306-318.
10. Gabriele J, Rajaram M, Zhang B, Sharma S, and Mishra R K. (2002). Modulation of a 40-kDa catecholamine-regulated protein following D-amphetamine treatment in discrete brain regions. Eur J Pharmacol. 453:13-19.
11. Gabriele J, Thomas N, N-Marandi S, and Mishra R K. (2007). Differential modulation of a 40 kDa catecholamine regulated protein in the core and shell subcompartments of the nucleus accumbens following chronic quinpirole and haloperidol administration in the rat. Syn. 61:835-842.
12. Gabriele J, Pontoriero G F, Thomas N, Thomson C A, Skoblenick K, Pristupa Z B, and Mishra R K. (2009). Cloning, characterization, and functional studies of a human 40-kDa catecholamine-regulated protein: implications in central nervous system disorders. Cell Stress Chaperones. 14(6):555-567.
13. Gabriele, J. et al. Cloning, characterization, and functional studies of a human 40-kDa catecholamine-regulated protein: implications in central nervous system disorders. Cell Stress. Chaperones 14, 555-567 (2009).
14. Gabriele, N., Pontoriero, G. F., Thomas, N., Shethwala, S. K., Pristupa, Z. B., Gabriele, J. P., 2010. Knockdown of mortalin within the medial prefrontal cortex impairs normal sensorimotor gating. Synapse. 64(11) 808-813.
15. Gill, S. S. et al. Direct brain infusion of glial cell line-derived neurotrophic factor in Parkinson disease. Nature Med. 9, 589-595 (2003).
16. Goto A, Doering L, Nair V D, and Mishra R K. (2001). Immunohistochemical localization of a 40-kDa catecholamine regulated protein in the nigrostriatal pathway. Brain Res. 900:314-319.
17. Hartl F U, and Hayer-Hartl M. (2009). Converging concepts of protein folding in vitro and in vivo. Nature Struct Mol Biol. 16:574-581.
18. Jin J, Hulette C, Wang Y, Zhang T, Pan C, and Wadhwa R. (2006). Proteomic identification of a Stress Protein, Mortalin/mthsp70/GRP75: relevance to Parkinson disease. Mol Cell Proteomics. 5:1193-1204.
19. Jin J, Li G J, Davis J, Zhu D, Wang Y, Pan C, and Zhang J. (2007). Identification of novel proteins associated with both alpha-synuclein and DJ-1. Mol Cell Proteomics. 6(5):845-859.
20. Kaul S C, Deocaris C C, and Wadhwa R. (2007). Three faces of mortalin: a housekeeper, guardian and killer. Exp Gerontol. 42(4):263-274.
21. Kiang J G, and Tsokos G C. (1998). Heat shock protein 70 kDa: molecular biology, biochemistry, and physiology. Pharmacol Ther. 80(2):183-201.
22. Kwiecien J M, O'Connor L T, Goetz B D, Delaney K H, Fletch A L, Duncan I D. Morphological and morphometric studies of the dysmyelinating mutant, the Long Evans shaker rat. J Neurocytol. August; 27(8):581-91 (1998).
23. Li H M, Niki T, Taira T, Iguchi-Ariga S M, and Ariga H. (2005). Association of DJ-1 with chaperones and enhanced association and colocalization with mitochondrial Hsp70 by oxidative stress. Free Radic Res. 39:1091-1099.
24. Liu Y, Liu W, Song X D, and Zuo J. (2005). Effect of GRP75/mthsp70/PBP74/mortalin overexpression on intracellular ATP level, mitochondrial membrane potential and ROS accumulation following glucose deprivation in PC 12 cells. Mol Cell Biochem. 268:45-51.
25. Malavolta, L., & Cabral, F. R. Peptides: Important tools for the treatment of central nervous disorders. Neuropeptides. 5, 309-316 (2011).
26. Mayer M P, Rudiger S, and Bukau B. (2000). Molecular basis for interactions of the DnaK chaperone with substrates. Biol Chem. 381:877-885.
27. Miyazaki I, and Asanuma M. (2009). Approaches to prevent dopamine quinone-induced neurotoxicity. Neurochem Res. 34(4):698-706.
28. Modi P I, Kashyap A, Nair V D, Ross G M, Fu M, Savelli J E, Marcotte E R, Barlas C, and Mishra R K. (1996). Modulation of brain catecholamine absorbing proteins by dopaminergic agents. Eur J Pharmacol. 299:213-220.
29. Modi, P. I. et al. Modulation of brain catecholamine absorbing proteins by dopaminergic agents. Eur. J. Pharmacol. 299, 213-220 (1996).
30. Nair V D, and Mishra R K. (2001). Molecular cloning, localization and characterization of a 40-kDa catecholamine-regulated protein. J Neurochem. 76:1142-1152.
31. Obeso J A, Rodriguez-Oroz M C, Goetz C G, Marin C, Kordower J H, Rodriguez M, Hirsch E C, Farrer M, Schapira A H, and Halliday G. (2010). Missing pieces in the Parkinson's disease puzzle. Nat Med.16:653-661.
32. Osorio C, Sullivan P M, He D N, Mace B E, Ervin J F, and Strittmatter W J. (2007). Mortalin is regulated by APOE in hippocampus of AD patients and by human APOE in TR mice. Neurobiol Aging. 28:1853-1862.
33. Pardridge, W. M. Re-engineering biopharmaceuticals for delivery to brain with molecular Trojan horses. Bioconjug. Chem. 19, 1327-1338 (2008).
34. Partridge, W. M., & Boardo, R. J. Pharmacokinetics and safety in rhesus monkeys of a monoclonal antibody-GDNF fusion protein for targeted blood-brain barrier delivery. Pharm. Res. 10, 2227-2236 (2009).
35. Rondou P, Haegeman G, and Van Craenenbroeck K. (2010). The dopamine D4 receptor: biochemical and signalling properties. Cell Mol Life Sci. 67:1971-1986.
36. Ross G M, McCarry B E, and Mishra R K. (1995). Covalent affinity labeling of brain catecholamine-absorbing proteins using a high-specific-activity substituted tetrahydronaphthalene. J Neurochem. 65:2783-2789.
37. Ross G M, McCarry B E, Thakur S, and Mishra R K. (1993). Identification of novel catecholamine absorbing proteins in the central nervous system. J Mol Neurosci. 4:141-148.
38. Schapira A H, and Jenner P. (2011). Etiology and pathogenesis of Parkinson's disease. Mov Disord. 26:1049-1055.
39. Schapira, A H. (2009). Neurobiology and treatment of Parkinson's disease. Trends Pharmacol Sci. 30:41-47.
40. Sharan N, Chong V Z, Nair V D, Mishra R K, Hayes R J, and Gardner E L. (2003). Cocaine treatment increases expression of a 40 kDa catecholamine-regulated protein in discrete brain regions. Syn. 47:33-44.
41. Sharan N, Nair V D, and Mishra R K. (2001). Modulation of a 40-kDa catecholamine regulated protein by dopamine receptor antagonists. Eur J Pharmacol. 413:73-79.
42. Shi M, Jin J, Wang Y, Beyer R P, Kitsou E, and Albin R L. (2008). Mortalin: a protein associated with progression of parkinson disease? J Neuropathol Exp Neurol. 67:117-124.
43. Söti C, Nagy E, Giricz Z, Vigh L, Csermely P, and Ferdinandy P. (2005). Heat shock proteins as emerging therapeutic targets. Br J Pharmacol. 146(6):769-780.
44. Thomson, C. A. & Ananthanarayanan, V. S. A method for expression and purification of soluble, active Hsp47, a collagen-specific molecular chaperone. Protein Exp. and Purif. 23, 8-13 (2001).
45. Van Laar V S, Dukes A A, Cascio M, and Hastings T G. (2008). Proteomic analysis of rat brain mitochondria following exposure to dopamine quinone: implications for Parkinson disease. Neurobiol Dis. 29:477-489.
46. Weiss M L, Medicetty S, Bledsoe A R, Rachakatla R S, Choi M, Merchav S, Luo Y, Rao M S, Velagaleti G, and Troyer D. (2006). Human umbilical cord matrix stem cells: preliminary characterization and effect of transplantation in a rodent model of Parkinson's disease. Stem Cells. 24(3):781-792.
47. Zhu X, Zhao X, Burkholder W F, Gragerov A, Ogata C M, Gottesman M E, and Hendrickson W A. (1996). Structural analysis of substrate binding by the molecular chaperone DnaK. Science 272: 1606-1614.

1. An isolated human CRP40 polypeptide fragment having a molecular weight of less than 30 kDa and comprising at least a functional portion of P2P4 (SEQ ID NO:5), or a functionally equivalent variant, fragment or derivative thereof.

2. The polypeptide of claim 1 which has a molecular weight of less than 29, 28, 27, 26, 25, 24, 23, 22, 21, 20, 19, 18, 17, 16, 15, 14, 13, 12, 11, 10, 9, 8, 7, 6, 5, 4, 3, 2 or 1 kDa.

3. The polypeptide of claim 1 or 2 which comprises at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 37, 33, 34, 35, 36, 37, 38, 39, 40, 41, 42, 43, 44, 45, 46, 47, 48, 49, 50, 51, 52, 53, 54, 55, 56, 57, 58, 59 or 60 contiguous amino acids of P2P4 (SEQ ID NO: 5).

4. The polypeptide of any one of claims 1 to 3 which comprises at least 10 contiguous amino acids of P2P4 (SEQ ID NO: 5).

5. The polypeptide of any one of claims 1 to 4 which comprises P1P4 (SEQ ID NO: 3), P1P5 (SEQ ID NO: 4), P2P4 (SEQ IDNO: 5) or P2P5 (SEQ ID NO: 6).

6. The polypeptide of any one of claims 1 to 5 which comprises P2P4 (SEQ ID NO: 5).

7. The polypeptide of any one of claims 1 to 5 which comprises a P2P4 fragment having an amino acid sequence selected from the group consisting of SEQ ID NOs: 9, 10, 11, 12, 13, 14, 15, 16, 17, 18, 19, 20, 21 and 22.

8. The polypeptide of any one of claims 1 to 7 wherein the functional variant has a sequence identity of at least 80%, 85%, 90%, 91%, 92%, 93%, 94%, 95%, 96%, 97%, 98% or 99%.

9. The polypeptide of any one of claims 1 to 8 wherein the functional variant has a sequence identity of at least 98%.

10. The polypeptide of any one of claims 1 to 8 wherein the functional variant comprises 1, 2, 3, 4, or 5 conservative modifications.

11. The polypeptide of claim 1 which consists of P2P4 (SEQ ID NO: 5).

12. The polypeptide of any one of claims 1 to 11 wherein the functional portion comprises all or part of a substrate binding region of CRP40.

13. The polypeptide of claim 12, wherein the substrate binding region comprises a phosphorylation site for PKC, PKA and/or CK1.

14. The polypeptide of any one of claims 1 to 13 wherein the polypeptide inhibits rotation in a 6-OHDA model by at least 25% compared to control when assessed at Day 4 post-administration.

15. The polypeptide of any one of claims 1 to 14 wherein the polypeptide inhibits rotation in a 6-OHDA model by at least 50% compared to control when assessed at Day 4 post-administration.

16. The polypeptide of any one of claims 1 to 15 wherein the polypeptide inhibits rotation in a 6-OHDA model by at least 80% compared to control when assessed at Day 4 post-administration.

17. The polypeptide of any one of claims 1 to 16 which does not bind dopamine.

18. A polypeptide encoded by a nucleic acid molecule having the nucleic acid sequence set forth in SEQ ID NO: 8; or a polynucleotide sequence with at least 80% sequence identity to SEQ ID NO:8 which hybridizes to the complement of SEQ ID NO:8 under stringent conditions.

19. A polypeptide encoded by a nucleic acid molecule having the nucleic acid sequence set forth in SEQ ID NO: 8

20. An isolated nucleic acid molecule encoding a polypeptide fragment as defined in any one of claims 1 to 19.

21. An isolated nucleic acid molecule selected from the group consisting of:
a) nucleic acid molecule comprising at least 15, 25, 30, 60, 75, 90, 105, 120, 135, 150, 165, or 180 contiguous nucleotides of the sequence set forth in any one of SEQ ID NOs: 8, 51, 52 and 53;
b) a fragment, variant or derivative of a) having at least at least 80%, 85%, 90%, 95%, 96%, 97%, 98% or 99% sequence identity thereto;
c) a nucleic acid molecule that hybridizes to the complement of the nucleic acid molecule of a) or b) under moderately stringent conditions; and
d) a nucleic acid molecule of a), b) or c) which encodes a functional CRP40 fragment.

22. The isolated nucleic acid molecule of claim 21 wherein the moderately stringent conditions comprise hybridization in 6× sodium chloride/sodium citrate (SSC) at 45° C., followed by one or more washes in 0.2×SSC, 0.1% SDS at 50-65° C.

23. The isolated nucleic acid molecule of claim 21 comprising at least 60 contiguous nucleotides of the sequence set forth in any one of SEQ ID NOs: 8, 51, 52 and 53 or a variant thereof having at least 80% sequence identity thereto and encoding a functional CRP40 fragment.

24. The isolated nucleic acid molecule of claim 21 comprising the sequence set forth in any one of SEQ ID NOs: 8, 51, 52 and 53.

25. The isolated nucleic acid molecule of claim 21 comprising at least 30 contiguous nucleotides of the sequence set forth in SEQ ID NO: 8.

26. The isolated nucleic acid molecule of claim 25 comprising a sequence as set forth in any one of SEQ ID NO: 32-45.

27. The isolated nucleic acid molecule of claim 25 comprising the sequence set forth in SEQ ID NO: 8.

28. A vector comprising the isolated nucleic acid molecule of claims 21 to 27.

29. The vector of claim 28 which is a pGEX-2T vector.

30. A cell comprising the vector of claim 28 or 29.

31. The cell of claim 30 which is a SHSY-5Y cell.

32. A primer comprising a polynucleotide consisting of at least 18 contiguous nucleotides of the nucleotide sequence of any one of SEQ ID NOS: 23-31 useful for preparing a CRP40 fragment.

33. The primer of claim 32 which consists of a nucleotide sequence selected from the group consisting of SEQ ID NO: 23-31.

34. A CRP40 polynucleotide prepared from any of the following primer pairs:
a) B1F and E5R;
b) B1F and E4R;
c) B2F and E5R;
d) B2F and E4R;
e) B3F and E4R; or
f) B3F and E5R.

35. A method of treating a neurological disorder in a subject in need thereof comprising administering to the subject a therapeutically effective amount of a CRP40 polypeptide fragment as defined in any one of claims 1-19 or a polynucleotide encoding a CRP40 polypeptide fragment as define in any one of claims 1-19.

36. The method of claim 35, comprising administering to the subject a therapeutically effective amount of a CRP40 polypeptide fragment as defined in any one of claims 1-19.

37. The method of claim 36, wherein the neurological disorder is characterized by one or more of:
(a) oxidative stress, mitochondrial dysfunction and/or abnormal protein folding;
(a) dopamine dysregulation; and
(c) movement impairment.

38. The method of claim 37, wherein the neurological disorder is characterized by one or more of oxidative stress, mitochondrial dysfunction and/or abnormal protein folding.

39. The method of claim 37, wherein the neurological disorder is characterized by dopamine dysregulation.

40. The method of claim 37, wherein the neurological disorder is characterized by movement impairment.

41. The method of claim any one of claims 35-37, wherein the neurological disorder is Parkinson's, a Parkinson-related disorder, tardive dyskinesia, drug-induced dyskinesia, cerebral ischemia, schizophrenia, bipolar disorder, an autistic disorder, Alzheimer's, Huntington's, ALS, ataxia telangiectasia, brain damage, dementia, diabetic neuropathy, dyslexia, dystonia, fetal alcohol syndrome, stroke, mini-stroke (transient ischemic attack), neurological sequelae of lupus, Neimann-Pick disease, Rett syndrome, sensory processing disorder, Tay-Sacs disease, Tourette syndrome, traumatic brain injury, Wilson's disease, Down's syndrome, multiple sclerosis, amyotrophic lateral sclerosis, hypoxia, ADHD or depression.

42. The method of claim 41, wherein the neurological disorder is Parkinson's disease, a Parkinson-related disorder, tardive dyskinesia or drug-induced dyskinesia.

43. The method of any one of claims 42, wherein the neurological disease is Parkinson's disease.

44. The method of any one of claim 42, wherein the neurological disorder a Parkinson-related disorder.

45. The method of any one of claim 44, wherein the Parkinson-related disorder is Lewy-body dementia or multiple systems atrophy.

46. The method of claim 42, wherein the neurological disorder is tardive dyskenisia or drug-induced dyskinesia.

47. The method of claim 46, wherein the drug-induced dyskinesia is L-dopa-induced or neuroleptic-induced.

48. A CRP40 fragment according to any one of claims 1 to 19 or a polynucleotide encoding a CRP fragment as define in any one of claims 1-19 for use in the treatment of a neurological disorder wherein the neurological disorder is as defined in any one of claims 37-47

49. The use of claim 48 wherein the neurological disorder is Parkinson's disease.

50. A pharmaceutical composition comprising a CRP40 polypeptide fragment as defined in any one of claims claims 1 to 19, or a polynucleotide encoding a polypeptide fragment as defined in any one of claims 1 to 19, and a pharmaceutically acceptable diluent or carrier.

51. An antibody against a CRP40 fragment as defined in any one of claims 1 to 19.
